(19) United States
(12) Patent Application Publication
(10) Pub. No.: US 2021/0008104 A1
(43) Pub. Date: Jan. 14, 2021

(54) COMPOSITIONS AND METHODS FOR INHIBITING HIV-1 REVERSE TRANSCRIPTASE

(71) Applicants: Samuel H. WILSON, Chapel Hill, NC (US); William A. BEARD, Chapel Hill, NC (US); David D. SHOCK, Apex, NC (US); The United States of America, as represented by the Secretary, Department of Health & Human Services, Rockville, MD (US)

(72) Inventors: Samuel H. Wilson, Chapel Hill, NC (US); William A. Beard, Chapel Hill, NC (US); David D. Shock, Apex, NC (US)

(21) Appl. No.: 16/637,092

(22) PCT Filed: Aug. 8, 2018
(86) PCT No.: PCT/US2018/045874

§ 371 Date: Feb. 6, 2020

Related U.S. Application Data
(60) Provisional application No. 62/542,600, filed on Aug. 8, 2017.

(51) Int. Cl. A61K 33/42; A61K 45/06

(52) U.S. Cl. CPC A61K 33/42; A61K 45/06

(57) ABSTRACT

The description provides compositions and methods of using a pyrophosphate analog, in which the bridging oxygen is replaced with an imido group (PNP) to increase the rate of the reverse polymerase reaction.

### CROSS-REFERENCE TO RELATED APPLICATIONS

The present application is a U.S. National Phase Application filed under 35 U.S.C. § 371, based on International PCT Patent Application No. PCT/US2018/045874, filed Aug. 8, 2018, which application claims priority to, and the benefit under 35 U.S.C. § 119(e) of, U.S. provisional patent application No. 62/542,600, filed Aug. 8, 2017. The entire teachings of each of which are incorporated herein by reference in their entirety.

### GOVERNMENT FUNDING

Research supporting this application was carried out by the United States of America as represented by the Secretary, Department of Health and Human Services.

### SEQUENCE LISTING

The instant application contains a Sequence Listing which has been filed electronically in ASCII format and is hereby incorporated by reference in its entirety. Said ASCII copy, created on May 14, 2020, is named 1420378_468US9_SL.txt and is 1,136 bytes in size.

### BACKGROUND

### 1. Field of the Discovery

The description provides compositions and methods of inhibition of nucleic acid amplification by HIV-1 Reverse Transcriptase (RT).

### 2. Background Information

DNA polymerases synthesize DNA during replication and repair of the genome. Accordingly, they are an attractive target for chemotherapies for uncontrolled cell growth; for example, cancer and viral infections. There are at least 17 human DNA polymerases, which utilize a common nucleotidyl transferase reaction wherein a deoxynucleoside triphosphate (dNTP) is added to the 3′ end of a growing DNA primer in a template-dependent manner. The reaction requires at least two divalent metal ions that facilitate an inline nucleophilic attack of the primer 3′-oxyanion on Pu of the incoming dNTP, resulting in extension of the primer strand by one nucleotide (i.e., dNMP) and pyrophosphate (PPi). This reaction is reversible, so that PPi and DNA can generate dNTP and a DNA primer strand that is one nucleotide shorter, in a process termed pyrophosphorolysis.

Although the forward DNA synthesis reaction is purposely favored, pyrophosphorolysis can be biologically important. Chainterminating nucleoside drugs are often used in an attempt to block DNA synthesis. However, drug resistance to chain-terminating agents can be correlated with the ability of stalled DNA polymerase to remove these nucleotides through pyrophosphorolysis. Additionally, pyrophosphorolysis can remove misinserted nucleotides opposite some DNA lesions as a proofreading activity, thereby increasing the fidelity of lesion bypass.

DNA polymerase β (pol β) is a model DNA polymerase for computational, structural, kinetic, and biological studies. The pyrophosphorolysis activity of pol β is highly dependent on the nature of the DNA substrate. For productive substrate binding in pyrophosphorolysis, the primer 3′ terminus must be bound in the nucleotide-binding pocket. In contrast, DNA synthesis requires that the primer terminus not occlude this site, but be situated at its boundary. These sites are termed the N site (nucleotide; i.e., postinsertion and pretranslocation) and P (primer) site. Structural studies indicate that the primer terminus is preferentially bound in the P site with one-nucleotide gapped DNA and in the N site with nicked DNA. Adding PPi-Mg2+ to crystals of binary complexes of pol β with nicked DNA generates a stable ternary product complex (pol-DNAnicked-PPi). Due to the unfavorable equilibrium for the reverse reaction, the level of the pol-DNAgap-dNTP complex would be beyond the limits of structural detection.

Here we have kinetically characterized pyrophosphorolysis and identified a PPi analog, imidodiphosphate (PNP), that alters the internal equilibrium, permitting structural characterization by time-lapse X-ray crystallography. Whereas pyrophosphorolysis was limited by a nonchemical step, replacing the bridging oxygen of PPi with an imido group resulted in a change in the rate-limiting step, so that the PNP-dependent reverse reaction was limited by chemistry. These results impact our mechanistic understanding of DNA polymerase nucleotidyl transferase chemistry and that key enzyme structural transitions can influence function.

Pyrophosphorolysis has been suggested to play a role in DNA polymerase fidelity and HIV-1 reverse transcriptase, as well as mitochondrial DNA polymerase γ, sensitivity to chain-terminating nucleoside drugs. An ongoing need exists for effective therapeutics for the treatment of diseases associated with undesired DNA replication, e.g., cancer and viral infection, such as HIV-1. As such, a better understanding of the reverse reaction is essential to define the overall reaction that will impact or modulate these proposed activities, and is a pre-requisite for rational drug design.

### SUMMARY

The present description relates to the kinetic characterization of pyrophosphorolysis and identification of a PPi analog, imidodiphosphate (PNP), that alters the internal equilibrium, permitting structural characterization by time-lapse X-ray crystallography. Whereas pyrophosphorolysis was limited by a nonchemical step, replacing the bridging oxygen of PPi with an imido group resulted in a change in the rate-limiting step, so that the PNP-dependent reverse reaction was limited by chemistry. These results impact our mechanistic understanding of DNA polymerase nucleotidyl transferase chemistry and that key enzyme structural transitions can influence function.

As such, the description provides a new approach to inhibit the DNA synthesis reaction of HIV-1 Reverse Transcriptase (RT). The DNA synthesis reaction by RT utilizes deoxynucleoside 5′-triphosphate (dNTP) as substrate, and like many other enzymes, the reaction is reversible. In the forward direction, elongated DNA and pyrophosphate (PPi) are the products, and in the reverse direction, dNTP and shortened DNA are the products.

Thus, in certain aspects the description provides compositions and methods including a pyrophosphate analogue, e.g., an analog of the reaction product, PPi. In certain embodiments, the analog is, e.g., imidodiphosphate (PNP). PNP was found to strongly promote the reverse reaction forming the dNTP product containing the PNP group, instead of the natural PPi group. This PNP-containing dNTP was found to be a potent inhibitor of the forward reaction by RT. An additional advantage is that drug resistant variants of RT that have enhanced reverse reactions will be more potently inhibited by an analogue as described herein.

In certain aspects and embodiments, the description provides therapeutic compositions comprising a pyrophosphate (PPi) analog, e.g., PNP. In certain embodiments, the compositions comprise an effective amount of a pyrophosphate (PPi) analog, e.g., PNP, and a pharmaceutically acceptable carrier.

In certain additional aspects and embodiments, the description provides a method of treating or ameliorating the symptoms of a disease or disorder comprising administering to a patient in need thereof, an effective amount of a composition comprising a pyrophosphate (PPi) analog, e.g., PNP, wherein the composition is effective in treating or ameliorating at least one symptom of the disease or disorder. In certain embodiments, the disease or disorder is a hyperproliferative disorder, a microbial-related disease or disorder, e.g., bacterial or viral infection. In certain embodiments the disease or disorder is cancer or HIV-1 infection.

The preceding general areas of utility are given by way of example only and are not intended to be limiting on the scope of the present disclosure and appended claims. Additional objects and advantages associated with the compositions, methods, and processes of the present invention will be appreciated by one of ordinary skill in the art in light of the instant claims, description, and examples. For example, the various aspects and embodiments of the invention may be utilized in numerous combinations, all of which are expressly contemplated by the present description. These additional advantages objects and embodiments are expressly included within the scope of the present invention. The publications and other materials used herein to illuminate the background of the invention, and in particular cases, to provide additional details respecting the practice, are incorporated by reference.

### BRIEF DESCRIPTION OF THE DRAWINGS

The accompanying drawings, which are incorporated into and form a part of the specification, illustrate several embodiments of the present invention and, together with the description, serve to explain the principles of the invention. The drawings are only for the purpose of illustrating an embodiment of the invention and are not to be construed as limiting the invention. Further objects, features and advantages of the invention will become apparent from the following detailed description taken in conjunction with the accompanying figures showing illustrative embodiments of the invention, in which:

FIG. 1. Single-turnover analysis of pyrophosphorolysis. FIG(a) Diagram illustrating the assay used to follow pyrophosphorolysis. A nicked DNA substrate utilizes pyrophosphate (PPi) to remove the 3′-[³²P] dCMP (C*) generating [α-32P] dCTP (dCTP*). A cold dCTP trap was included in the reaction to prevent insertion of the radioactive product and to regenerate nicked DNA with an unlabeled 3′-terminus. Product formation (dCTP*) was monitored by thin-layer chromatography (TLC). FIG (b) Data points, time, and ligand concentrations were selected to provide full coverage; i.e., multiple points were collected below and above reaction half-times (≥6 time points) and ligand-binding affinities (≥5 concentrations), respectively. Time courses were fit to a single exponential (gray lines). FIG (c) A secondary plot of the PPi concentration dependence of the observed first-order rate constants (kobs). These data were fit to a hyperbola (equation (1) in Online Methods, black line) to derive krev and Kd (Supplementary Table 1). FIG (d) Simplified kinetic scheme for a DNA polymerase single-nucleotide insertion reaction. The chemical step (K4) is flanked by enzyme conformational changes (K3 and K5). Ligand binding (K1, K2, K6, and K7) occurs to one form of the enzyme (circles) that undergoes a nonchemical conformational change to an alternate form (squares). These conformational (conf.) states are often described as open or closed forms of the polymerase, respectively

FIG. 2. Qualitative assay of pol β reverse reaction with various PPi analogs. Pol β was pre-incubated with 5′-³²P-labeled nicked DNA substrate for 5 min at 37° C. and mixed with MgCl2 and PPi or an analog. The final concentrations of MgCl2 and PPi (analog) were 10 and 1 mM, respectively. The full gel is shown in FIG. 13a. The reverse reaction generates products shorter that the 16-mer primer. The structures of PPi, imidodiphosphate (PNP), and three bisphosphonates (clodronate, etidronate, and pamidronate) surveyed are shown above the gel image.

FIG. 3. Single-turnover analysis of PNP-dependent reverse reaction. FIG(a) Diagram illustrating the assay used to follow the reverse reaction. A nicked DNA substrate utilizes PNP to remove 3′-[³²P] dCMP (C*) generating [α-32P] dCMPPNP (dCMPPNP*). A cold dCTP trap was included in the reaction to prevent insertion of the radioactive product and to regenerate nicked DNA with an unlabeled 3′-terminus. Product formation (dCMPPNP*) was monitored by TLC. FIG (b) Data points, time, and ligand concentrations were selected to provide full coverage; i.e., multiple points were collected below and above reaction half-times (≥6 time points) and ligand binding affinities (≥5 concentrations), respectively. Time courses were fit to a single exponential (gray lines). FIG (c) A secondary plot of the PNP concentration dependence of the observed first-order rate constants (kobs). These data were fit to a hyperbola (equation (1)) to derive krev and Kd (Supplementary Table 1).

FIG. 4. Removal of aberrant primer termini by pol β-dependent reverse reaction. FIG(a) Pol β and one-nucleotide gapped DNA were mixed with MgCl2 and various triphosphates of chain-terminating nucleotides (ddCTP, AZTTP, araCTP, or dFdCTP) as outlined in Online Methods. The gap-filling reaction generated a nicked DNA substrate. The reverse reaction was initiated by addition of MgCl2 and PPi or PNP. After 3 min, an aliquot was removed, quenched, and analyzed on a denaturing gel. The 15-mer primer (P), 16-mer terminated nicked DNA substrate (ddCMP, AZTMP, araCMP, or dFdCMP) and reverse reaction products (<16-mer) are indicated. The full gel is shown in FIG. 13b. FIG (b) Pol β was pre-incubated with 5′-32P-labeled nicked DNA substrate with a matched (G-C) or mismatched (G-A or G-T) primer terminal base pair and mixed with mM MgCl2 and PPi or PNP. The 16-mer substrate and reverse reaction products (<16-mer) are indicated. The full gel is shown in FIG. 13c. T-P, Template-primer; O and N refer to the identity of the phosphate bridging atom in P—X—P.

FIG. 5. Observing the reverse reaction by time-lapse crystallography. FIG(a-d) The pol β active site is shown with key residues indicated; all Fo-Fc omit maps are contoured at 36 (green). Metal coordination and key distances (Å) are indicated with dashed lines. The carbons of the terminal base pair of the nicked DNA are yellow. The carbons of the upstream DNA are gray. The primer nucleotide upstream of the primer terminus (P10), as well as PNP are indicated. The bridging nitrogen of PNP is colored blue. FIG(a) The active site for the ground-state nicked DNA substrate complex with PNP and Ca2+(orange; c, catalytic; n, nucleotide) is shown. The amino-terminal end of u-helix N (helix-N) is also illustrated. FIG(b) An overlay of the substrate nicked DNA-PNP-Ca2+ complex (yellow carbons) and the nicked DNA-PPi-Mn2+ product complex (PDB code 4KLH; light blue carbons) is shown. The manganese atom from the PPi complex is purple. FIG(c) A close-up of the PPi and PNP phosphate groups from b. The arrows indicate the phosphate oxygen shift for PNP relative to PPi. The distance between the phosphate and the attacking oxygen for PNP and PPi is indicated with a dashed line. FIG(d) The reactant complex for the reverse reaction is shown following a short MgCl2 soak. The Mg2+ and water ions are shown as red and blue spheres, respectively. The distances between the bridging water, Arg183, and the nitrogen of PNP are indicated. The catalytic and nucleotide-binding metals are labeled as Mgc and Mgn, respectively. FIG(e) The final one-nucleotide gapped DNA-dCMPPNP ternary complex is shown following the reverse reaction.

FIG. 6. The pyrophosphate analog imidodiphosphate (PNP) alters the reaction equilibrium of human DNA polymerase β, and the resulting increase in the rate of pyrophosphorolysis enables kinetic and structural dissection of this reverse reaction of the enzyme.

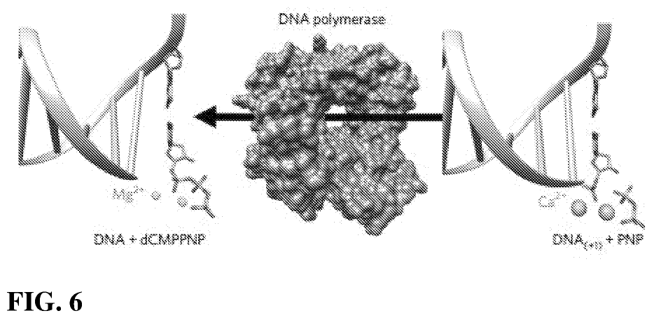

FIG. 7.|Thio-elemental effect on pyrophosphorolysis. (a) Diagram illustrating the assay used to follow pyrophosphorolysis. On a nicked DNA substrate, pol β utilizes PPi to remove the 3′-[³²P]dAMP or 3′-[³⁵S]dAMP (A*) generating [α-³²P]dATP or [α-³⁵S]dATP (dATP*), respectively. A cold dATP or dATP(aS) trap was included in the reaction to prevent insertion of the radioactive product and to regenerate nicked DNA with an unlabeled 3′-terminus. Product formation (dATP*) was monitored by TLC. (b) Image of the exposed TLC plate for formation of [α-³²P]dATP. Lane M is [α-³²P]dATP alone. An image of the full plate is shown in FIG. 14a. (c) Pol β-dependent dATP* formation in the presence of 1 mM PPi with a 3′-[32P]dAMP (▪) or 3′-[5S]dAMP (□) primer terminus. Single-turnover time courses were fit to a single exponential (solid and dashed gray lines for ³²P- and ³⁵S-labeled dATP, respectively) (k_{obs}=0.030/s and 0.039/s for removal of ³²P- and ³⁵S-labeled dAMP, respectively).

FIG. 8.|Pyrophosphate exchange. (a) The exchange reaction follows the movement of radioactive-label in [32P]PPᵢ into dNTP to distinguish whether PPi binding occurs prior to (upper panel) or following (lower panel) a rate-limiting conformational change (red arrow)⁵⁰. In this experiment, the ternary product complex was generated in situ (unlabeled dNTP is present to generate nicked DNA and cold PPᵢ, gray labels) under single-turnover conditions (pol>>DNA) and the rate of radioactive movement from labeled PPᵢ into dNTP (blue) was measured. These schemes illustrate that if PPᵢ binding occurs prior to the slow conformational change, then the measured rate of pyrophosphorolysis will be similar to the rate of exchange. In contrast, if PPᵢ binding occurs after the slow conformational change, then the rate of exchange (rapid PPᵢ binding and chemistry) will be faster than the measured rate of pyrophosphorolysis. (b) Pol β was pre-incubated with unlabeled nicked DNA and mixed with a solution containing [³²P]PP; and cold dCTP. Radioactive dCTP was followed by TLC. The solid line represents the best fit to a linear equation. The observed rate for the exchange reaction (slope/enzyme-DNA complex) was 0.028/s. Since the rate of PPᵢ exchange as determined by substrate cycling (i.e., alternating nucleotide insertion and removal) is similar to that measured by single-turnover analysis, PPᵢ binding occurs prior to the conformational change. Since the rate of PPᵢ exchange as determined by substrate cycling (i.e., alternating nucleotide insertion and removal) is similar to that measured by single-turnover analysis, PP binding occurs prior to the conformational change.

FIG. 9.|PNP-induced gap-filling reaction. (a) Diagram illustrating the assay used to follow PNP-induced gap-filling DNA synthesis. An unlabeled nicked DNA substrate with two deoxycytidine residues at the 3′-primer terminus was incubated with a low concentration of PNP as described in Online Methods. A single-nucleotide gapped DNA substrate (G in the gap) with a 5′-6-FAM (*) 15-mer labeled primer (P) was then mixed with this solution to determine if complementary deoxynucleoside triphosphates (i.e., dCMPPNP) were generated in the initial reaction that could be used to fill the gap. (b) Substrate/products were resolved on a denaturing gel and visualized by phosphorimaging. Gap-filling DNA synthesis generates a 16-mer product, while pyrophosphorolysis creates a 14-mer product. An image of the full gel is shown in FIG. 14b.

FIG. 10.|Thio-elemental effect on PNP-dependent reverse reaction. (a) Diagram illustrating the assay used to follow PNP-dependent reverse reaction. A nicked DNA substrate utilizes PNP to remove a 3′-[32P]dAMP or 3′-[³⁵S]dAMP (A*) generating [α-³²P]dAMPPNP or [α-³⁵S]dAMPPNP (dATP*), respectively. A cold dATP trap was included in the reaction to prevent insertion of the radioactive product and to regenerate nicked DNA with an unlabeled 3′-terminus. Product formation (dATP*) was monitored by TLC. (b) A secondary plot of the PNP concentration dependence of the observed first-order rate constants (k_{obs}) for single-turnover time courses for the removal of a 3′-[32P]dAMP in nicked DNA. These data were fit to a hyperbola (Eq. 1, gray line) to derive kᵣₑᵥ and K_{d} (Supplementary Table 1). (c) A secondary plot of the PNP concentration dependence of the observed first-order rate constants (k_{obs}) for single-turnover time courses for the removal of a 3′-[³⁵S]dAMP in nicked DNA. The duplicate points at 1000 μM PNP represents data from independent experiments. These data were fit to a hyperbola (Eq. 1, gray line) to derive kᵣₑᵥ and K_{d} (Supplementary Table 1).

FIG. 11.|Single-turnover analysis for gap filling insertion with dGMPPNP. (a) Pol β-dependent single-nucleotide gap filling DNA synthesis with 0.1 μM (▴), 0.2 μM (⋄), 0.5 M (♦), 1 μM (□), 2 μM (▪), 4 μM (◯) and 5 μM (●) dGMPPNP. Time courses were fit to a single exponential (gray lines). (b) A secondary plot of the dGMPPNP concentration dependence of the observed first-order rate constants (k_{obs}). These data were fit to a hyperbola (Eq. 1, gray line) to derive kₚₒₗ and K_{d} (Supplementary Table 1).

FIG. 12.|Equilibrium analysis of pol β bound with one-nucleotide gapped and nicked DNA. (a) Image of a representative sequencing gel showing the time dependence of single-nucleotide gap filling in the presence of 20, 50 or 100 M PNP. An image of the full gel is shown in FIG. 14c. In this assay, the 5′-labeled primer (15-mer) can be extended one nucleotide (16-mer). The first lane includes primer only. (b) Quantification of the gel shown in panel a indicating that equilibrium had been established (i.e., concentration of DNA product does not change with time, 30-80 s) and that the amount of product is sensitive to the concentration of PNP (▪, 20 μM; ●, 50 μM; ♦, 100 μM). The calculated equilibrium constants are 1.5, 1.9, and 2.2 for 20, 50 and 100 M PNP, respectively. (c) Quantification of an assay with PPi indicating that equilibrium had been established and that the amount of product is weakly sensitive to the concentration of PPᵢ (▪, 1000 μM; ♦, 2000 M). The calculated equilibrium constants are 62,700 and 82,300 for 1000 and 2000 M PPᵢ, respectively.

FIG. 13.|Full gel images. The cropped image in the respective figures is indicated. (a) FIG. 2. (b) FIG. 4a. (c) FIG. 4b.

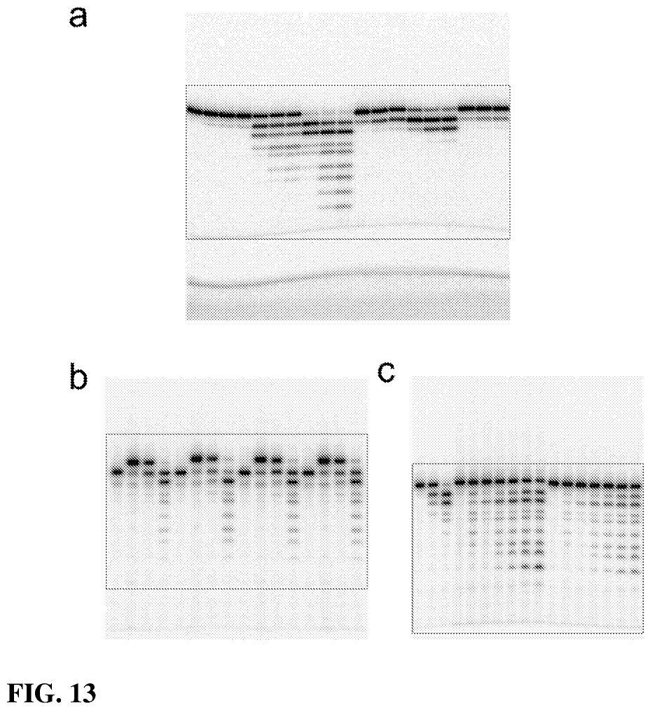

FIG. 14.|Full TLC plate or gel images. The cropped image in the respective figures is indicated. (a) FIG. 7b. (b) FIG. 9b. (c) FIG. 12a.

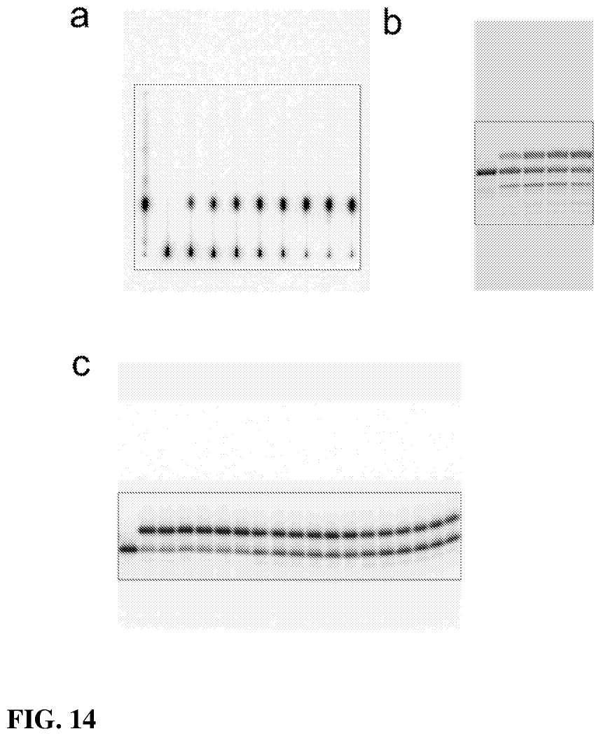

FIG. 15. Supplementary Table 1. Summary of kinetic parameter.

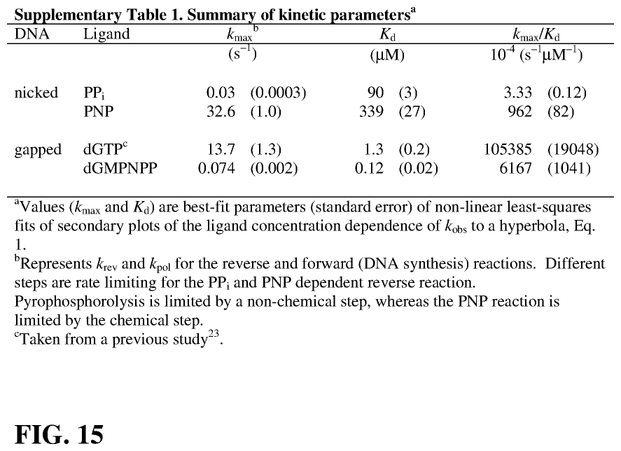

FIG. 16. Supplementary Table 2. Data collection and refinement statistic.

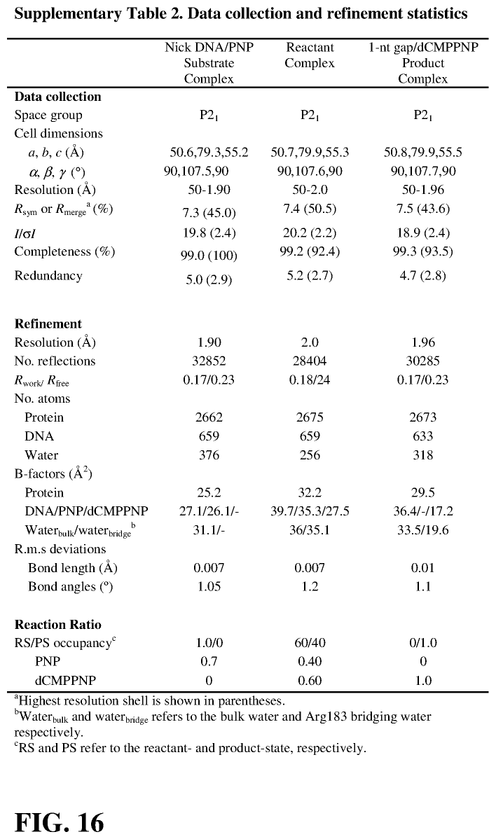

### DETAILED DESCRIPTION

The following is a detailed description provided to aid those skilled in the art in practicing the present invention. Those of ordinary skill in the art may make modifications and variations in the embodiments described herein without departing from the spirit or scope of the present disclosure. All publications, patent applications, patents, figures and other references mentioned herein are expressly incorporated by reference in their entirety.

DNA polymerases catalyze efficient and high-fidelity DNA synthesis. While this reaction favors nucleotide incorporation, polymerases also catalyze a reverse reaction, pyrophosphorolysis, that removes the DNA primer terminus and generates deoxynucleoside triphosphates. Because pyrophosphorolysis can influence polymerase fidelity and sensitivity to chain-terminating nucleosides, we analyzed pyrophosphorolysis with human DNA polymerase β and found the reaction to be inefficient. The lack of a thio-elemental effect indicated that this reaction was limited by a nonchemical step. Use of a pyrophosphate analog, in which the bridging oxygen is replaced with an imido group (PNP), increased the rate of the reverse reaction and displayed a large thio-elemental effect, indicating that chemistry was now rate determining. Time-lapse crystallography with PNP captured structures consistent with a chemical equilibrium favoring the reverse reaction. These results highlight the importance of the bridging atom between the β- and γ-phosphates of the incoming nucleotide in reaction chemistry, enzyme conformational changes, and overall reaction equilibrium.

The present description relates to the kinetic characterization of pyrophosphorolysis and identification of a PPᵢ analog, imidodiphosphate (PNP), that alters the internal equilibrium, permitting structural characterization by time-lapse X-ray crystallography. Whereas pyrophosphorolysis was limited by a nonchemical step, replacing the bridging oxygen of PPᵢ with an imido group resulted in a change in the rate-limiting step, so that the PNP-dependent reverse reaction was limited by chemistry. These results impact our mechanistic understanding of DNA polymerase nucleotidyl transferase chemistry and that key enzyme structural transitions can influence function.

As such, the description provides a new approach to inhibit the DNA synthesis reaction of HIV-1 Reverse Transcriptase (RT). The DNA synthesis reaction by RT utilizes deoxynucleoside 5′-triphosphate (dNTP) as substrate, and like many other enzymes, the reaction is reversible. In the forward direction, elongated DNA and pyrophosphate (PPi) are the products, and in the reverse direction, dNTP and shortened DNA are the products.

Thus, in certain aspects the description provides compositions and methods including a pyrophosphate analogue, e.g., an analog of the reaction product, PPi. In certain embodiments, the analog is, e.g., imidodiphosphate (PNP). PNP was found to strongly promote the reverse reaction forming the dNTP product containing the PNP group, instead of the natural PPi group. This PNP-containing dNTP was found to be a potent inhibitor of the forward reaction by RT. An additional advantage is that drug resistant variants of RT that have enhanced reverse reactions will be more potently inhibited by an analogue as described herein.

In certain aspects and embodiments, the description provides therapeutic compositions comprising a pyrophosphate (PPi) analog, e.g., PNP. In certain embodiments, the compositions comprise an effective amount of a pyrophosphate (PPi) analog, e.g., PNP, and a pharmaceutically acceptable carrier.

In certain additional aspects and embodiments, the description provides a method of treating or ameliorating the symptoms of a disease or disorder comprising administering to a patient in need thereof, an effective amount of a composition comprising a pyrophosphate (PPi) analog, e.g., PNP, wherein the composition is effective in treating or ameliorating at least one symptom of the disease or disorder. In certain embodiments, the disease or disorder is a hyperproliferative disorder, a microbial-related disease or disorder, e.g., bacterial or viral infection. In certain embodiments the disease or disorder is cancer or HIV-1 infection.

Unless otherwise defined, all technical and scientific terms used herein have the same meaning as commonly understood by one of ordinary skill in the art to which this invention belongs. The terminology used in the description is for describing particular embodiments only and is not intended to be limiting of the invention.

Where a range of values is provided, it is understood that each intervening value, to the tenth of the unit of the lower limit unless the context clearly dictates otherwise (such as in the case of a group containing a number of carbon atoms in which case each carbon atom number falling within the range is provided), between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

The following terms are used to describe the present invention. In instances where a term is not specifically defined herein, that term is given an art-recognized meaning by those of ordinary skill applying that term in context to its use in describing the present invention.

The articles “a” and “an” as used herein and in the appended claims are used herein to refer to one or to more than one (i.e., to at least one) of the grammatical object of the article unless the context clearly indicates otherwise. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

As used herein in the specification and in the claims, the phrase “at least one,” in reference to a list of one or more elements, should be understood to mean at least one element selected from anyone or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a nonlimiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, in certain methods described herein that include more than one step or act, the order of the steps or acts of the method is not necessarily limited to the order in which the steps or acts of the method are recited unless the context indicates otherwise.

The term “compound”, as used herein, unless otherwise indicated, refers to any specific chemical compound disclosed herein and includes tautomers, regioisomers, geometric isomers, and where applicable, stereoisomers, including optical isomers (enantiomers) and other steroisomers (diastereomers) thereof, as well as pharmaceutically acceptable salts and derivatives (including prodrug forms) thereof where applicable, in context. Within its use in context, the term compound generally refers to a single compound, but also may include other compounds such as stereoisomers, regioisomers and/or optical isomers (including racemic mixtures) as well as specific enantiomers or enantiomerically enriched mixtures of disclosed compounds. The term also refers, in context to prodrug forms of compounds which have been modified to facilitate the administration and delivery of compounds to a site of activity. It is noted that in describing the present compounds, numerous substituents and variables associated with same, among others, are described. It is understood by those of ordinary skill that molecules which are described herein are stable compounds as generally described hereunder. When the bond is shown, both a double bond and single bond are represented within the context of the compound shown.

The term “patient” or “subject” is used throughout the specification to describe an animal, preferably a human or a domesticated animal, to whom treatment, including prophylactic treatment, with the compositions according to the present invention is provided. For treatment of those infections, conditions or disease states which are specific for a specific animal such as a human patient, the term patient refers to that specific animal, including a domesticated animal such as a dog or cat or a farm animal such as a horse, cow, sheep, etc. In general, in the present invention, the term patient refers to a human patient unless otherwise stated or implied from the context of the use of the term.

The term “effective” is used to describe an amount of a compound, composition or component which, when used within the context of its intended use, effects an intended result. The term effective subsumes all other effective amount or effective concentration terms, which are otherwise described or used in the present application.

The terms “nucleic acid,” “polynucleotides,” and “oligonucleotides” refers to biopolymers of nucleotides and, unless the context indicates otherwise, includes modified and unmodified nucleotides, and both DNA and RNA. For example, in certain embodiments, the nucleic acid is a peptide nucleic acid (PNA). Typically, the methods as described herein are performed using DNA as the nucleic acid template for amplification. However, nucleic acid whose nucleotide is replaced by an artificial derivative or modified nucleic acid from natural DNA or RNA is also included in the nucleic acid of the present invention insofar as it functions as a template for synthesis of complementary chain. The nucleic acid of the present invention is generally contained in a biological sample. The biological sample includes animal, plant or microbial tissues, cells, cultures and excretions, or extracts therefrom. In certain aspects, the biological sample includes intracellular parasitic genomic DNA or RNA such as virus or mycoplasma. The nucleic acid may be derived from nucleic acid contained in said biological sample. For example, genomic DNA, or cDNA synthesized from mRNA, or nucleic acid amplified on the basis of nucleic acid derived from the biological sample, are preferably used in the described methods.

“Complementarity” refers to the ability of a nucleic acid to form hydrogen bond(s) or hybridize with another nucleic acid sequence by either traditional Watson-Crick or other non-traditional types. As used herein “hybridization,” refers to the binding, duplexing, or hybridizing of a molecule only to a particular nucleotide sequence under low, medium, or highly stringent conditions, including when that sequence is present in a complex mixture (e.g., total cellular) DNA or RNA. See e.g. Ausubel, et al., CURRENT PROTOCOLS IN MOLECULAR BIOLOGY, John Wiley & Sons, New York, N.Y., 1993

If a nucleotide at a certain position of a polynucleotide is capable of forming a Watson-Crick pairing with a nucleotide at the same position in an anti-parallel DNA or RNA strand, then the polynucleotide and the DNA or RNA molecule are complementary to each other at that position. The polynucleotide and the DNA or RNA molecule are “substantially complementary” to each other when a sufficient number of corresponding positions in each molecule are occupied by nucleotides that can hybridize or anneal with each other in order to effect the desired process. A complementary sequence is a sequence capable of annealing under stringent conditions to provide a 3′-terminal serving as the origin of synthesis of complementary chain.

The term “template” used in the present invention means nucleic acid serving as a template for synthesizing a complementary chain in a nucleic acid amplification technique. A complementary chain having a nucleotide sequence complementary to the template has a meaning as a chain corresponding to the template, but the relationship between the two is merely relative. That is, according to the methods described herein a chain synthesized as the complementary chain can function again as a template. That is, the complementary chain can become a template. In certain embodiments, the template is derived from a biological sample, e.g., plant, animal, virus, micro-organism, bacteria, fungus, etc. In certain embodiments, the animal is a mammal, e.g., a human patient.

“Patient sample” refers to any sample taken from a patient and can include blood, stool, swabs, sputum, Broncho Alveolar Lavage Fluid, tissue samples, urine or spinal fluids. Other suitable patient samples and methods of extracting them are well known to those of skill in the art. A “patient” or “subject” from whom the sample is taken may be a human or a non-human animal. When a sample is not specifically referred to as a patient sample, the term also comprises samples taken from other sources. Examples include swabs from surfaces, water samples (for example waste water, marine water, lake water, drinking water), food samples, cosmetic products, pharmaceutical products, fermentation products, cell and micro-organism cultures and other samples in which the detection of a micro-organism is desirable.

In the present invention, the terms “synthesis” and “amplification” of nucleic acid are used. The synthesis of nucleic acid in the present invention means the elongation or extension of nucleic acid from an oligonucleotide serving as the origin of synthesis. If not only this synthesis but also the formation of other nucleic acid and the elongation or extension reaction of this formed nucleic acid occur continuously, a series of these reactions is comprehensively called amplification.

In the present specification, the simple expression “5′-side” or “3′-side” refers to that of a nucleic acid chain serving as a template, wherein the 5′ end generally includes a phosphate group and a 3′ end generally includes a free —OH group.

The term “disease state or condition” is used to describe any disease state or condition, in particular, cancers, including those relating to genetic abnormalities, or due to the presence of a pathogenic organism such as a virus, bacteria, archae, protozoa or multicellular organism.

The target template used in the present invention may be any polynucleic acid that comprises suitable primer binding regions that allow for amplification of a polynucleic acid of interest. The skilled person will understand that the forward and reverse primer binding sites need to be positioned in such a manner on the target template that the forward primer binding region and the reverse primer binding region are positioned 5′ of the sequence which is to be amplified on the sense and antisense strand, respectively.

The target template may be single or double stranded. Where the target template is a single stranded polynucleic acid, the skilled person will understand that the target template will initially comprise only one primer binding region. However, the binding of the first primer will result in synthesis of a complementary strand which will then contain the second primer binding region.

Examples of techniques sufficient to direct persons of skill through in vitro amplification methods are found in Berger, supra, Sambrook, supra, and Ausubel, supra, as well as Mullis, et al., U.S. Pat. No. 4,683,202 (1987); and Innis, et al., PCR Protocols A Guide to Methods and Applications, Eds., Academic Press Inc., San Diego, Calif. (1990). Commercially available kits for genomic PCR amplification are known in the art. See, e.g., Advantage-GC Genomic PCR Kit (Clontech). Additionally, e.g., the T4 gene 32 protein (Boehringer Mannheim) can be used to improve yield of long PCR products.

The pathogenic organism to be treated may be any micro-organisms, such as viruses, bacteria, mycoplasma and fungi. The micro-organism can be pathogenic but it may also be a non-pathogenic micro-organism. The microorganism may also be a genetically modified organism (GMO). Furthermore, the methods of the present invention can be used to identify genetically modified crops and animals, for the detection of a disease state; for the prediction of an adverse reaction from a therapy and also for the prediction of a disease state susceptibility.

In certain embodiments, the microbe is a bacterium. In certain embodiments, the bacteria is a member of a genus selected from the group consisting of Bacillus, Bartonella, Bordetella, Borrelia, Brucella, Campylobacter, Chlamydia, Chlamydophila, Clostridium, Corynebacterium, Enterococcus, Escherichia, Francisella, Haemophilus, Legionella, Leptospira, Listeria, Mycobacterium, Mycoplasma, Neisseria, Pseudomonas, Rickettsia, Salmonella, Shigella, Staphylococcus, Streptococcus, Treponema, Ureaplasma, Vibrio, and Yershinia.

In certain embodiments, the bacteria is a member of the group consisting of Bacillus anthracis, Bacillus cereus, Bartonella henselae, Bartonella Quintana, Bordetella pertussis, Borrelia burgdorferi, Borrelia garinii, Borrelia afzelii, Borrelia recurrentis, Brucella abortus, Brucella canis, Brucella melitensis, Brucella suis, Campylobacter jejuni, Chlamydia pneumonia, Chlamydia trachomatis, Chlamydophila psittaci, Clostridium botulinum, Clostridium difficile, Clostridium perfringens, Clostridium tetani, Corynebacterium diphtheria, Enterococcus faecalis, Enterococcus faecium, Escherichia coli, Francisella tularensis, Haemophilus influenza, Helicobacter pylori, Legionella pneumophila, Leptospira interrogans, Leptospira santarosai, Leptospira weilii, Leptospira noguchii, Listeria monocytogenes, Mycobacterium leprae, Mycobacterium tuberculosis, Mycobacterium ulcerans, Mycoplasma pneumonia, Neisseria gonorrhoeae, Neisseria meningitides, Pseudomonas aeruginosa, Rickettsia rickettsia, Salmonella typhi, Salmonella typhimurium, Shigella sonnei, Staphylococcus aureus, Staphylococcus epidermidis, Staphylococcus saprophyticus, Streptococcus agalactiae, Streptococcus pneumonia, Streptococcus pyogenes, Treponema pallidum, Ureaplasma urealyticum, Vibrio cholera, Yersinia pestis, Yersinia enterocolitica, and Yersinia pseudotuberculosis.

In certain embodiments, the target nucleic acid template is from tubercle bacillus (MTB or TB). In certain additional embodiments, the target nucleic acid template is from the rpoB gene from MTB. In still further embodiments, the target nucleic acid template is rpoB13.5 F6.

In certain embodiments, the virus is a member of a family selected from the group consisting of Adenoviridae, Herpesviridae, Papillomaviridae, Polyomaviridae, Poxviridae, Hepadnaviridae, Parvoviridae, Astroviridae, Caliciviridae, Picornaviridae, Coronaviridae, Flaviviridae, Togaviridae, Hepeviridae, Retroviridae, Orthomyxoviridae, Arenaviridae, Bunyaviridae, Filoviridae, Paramyxoviridae, Rhabdoviridae, and Reoviridae.

In certain embodiments, the virus is a member selected from the group consisting of Adenovirus, Herpes simplex type 1, Herpes simplex type 2, Varicella-zoster virus, Epstein-Barr virus, Human cytomegalovirus, Human herpesvirus type 8, Human papillomavirus, BK virus, JC virus, Smallpox, Hepatitis B, Human bocavirus, Parvovirus B19, Human astrovirus, Norwalk virus, coxsackievirus, hepatitis A virus, poliovirus, rhinovirus, Severe acute respiratory syndrome virus, Hepatitis C virus, yellow fever virus, dengue virus, West Nile virus, Rubella virus, Hepatitis E virus, Human immunodeficiency virus (HIV), Influenza virus, Guanarito virus, Junin virus, Lassa virus, Machupo virus, Sabiá virus, Crimean-Congo hemorrhagic fever virus, Ebola virus, Marburg virus, Measles virus, Mumps virus, Parainfluenza virus, Respiratory syncytial virus, Human metapneumovirus, Hendra virus, Nipah virus, Rabies virus, Hepatitis D, Rotavirus, Orbivirus, Coltivirus, and Banna virus.

In one aspect, this invention relates to pharmaceutical compositions containing one or more compounds of the present invention. These compositions can be utilized to achieve the desired pharmacological effect by administration to a patient in need thereof. A patient, for the purpose of this invention, is a mammal, including a human, in need of treatment for the particular condition or disease. Therefore, the present invention includes pharmaceutical compositions that are comprised of a pharmaceutically acceptable carrier and a pharmaceutically effective amount of a compound, or salt thereof, of the present invention. A pharmaceutically acceptable carrier is preferably a carrier that is relatively non-toxic and innocuous to a patient at concentrations consistent with effective activity of the active ingredient so that any side effects ascribable to the carrier do not vitiate the beneficial effects of the active ingredient. A pharmaceutically effective amount of a compound is preferably that amount which produces a result or exerts an influence on the particular condition being treated. The compounds of the present invention can be administered with pharmaceutically-acceptable carriers well known in the art using any effective conventional dosage unit forms, including immediate, slow and timed release preparations, orally, parenterally, topically, nasally, ophthalmically, optically, sublingually, rectally, vaginally, and the like.

For oral administration, the compounds can be formulated into solid or liquid preparations such as capsules, pills, tablets, troches, lozenges, melts, powders, solutions, suspensions, or emulsions, and may be prepared according to methods known to the art for the manufacture of pharmaceutical compositions. The solid unit dosage forms can be a capsule that can be of the ordinary hard- or soft-shelled gelatin type containing, for example, surfactants, lubricants, and inert fillers such as lactose, sucrose, calcium phosphate, and corn starch.

In another embodiment, the compounds of this invention may be tableted with conventional tablet bases such as lactose, sucrose and cornstarch in combination with binders such as acacia, corn starch or gelatin, disintegrating agents intended to assist the break-up and dissolution of the tablet following administration such as potato starch, alginic acid, corn starch, and guar gum, gum tragacanth, acacia, lubricants intended to improve the flow of tablet granulation and to prevent the adhesion of tablet material to the surfaces of the tablet dies and punches, for example talc, stearic acid, or magnesium, calcium or zinc stearate, dyes, coloring agents, and flavoring agents such as peppermint, oil of wintergreen, or cherry flavoring, intended to enhance the aesthetic qualities of the tablets and make them more acceptable to the patient. Suitable excipients for use in oral liquid dosage forms include dicalcium phosphate and diluents such as water and alcohols, for example, ethanol, benzyl alcohol, and polyethylene alcohols, either with or without the addition of a pharmaceutically acceptable surfactant, suspending agent or emulsifying agent. Various other materials may be present as coatings or to otherwise modify the physical form of the dosage unit. For instance tablets, pills or capsules may be coated with shellac, sugar or both.

Dispersible powders and granules are suitable for the preparation of an aqueous suspension. They provide the active ingredient in admixture with a dispersing or wetting agent, a suspending agent and one or more preservatives. Suitable dispersing or wetting agents and suspending agents are exemplified by those already mentioned above. Additional excipients, for example those sweetening, flavoring and coloring agents described above, may also be present.

The pharmaceutical compositions of this invention may also be in the form of oil-in-water emulsions. The oily phase may be a vegetable oil such as liquid paraffin or a mixture of vegetable oils. Suitable emulsifying agents may be (1) naturally occurring gums such as gum acacia and gum tragacanth, (2) naturally occurring phosphatides such as soy bean and lecithin, (3) esters or partial esters derived form fatty acids and hexitol anhydrides, for example, sorbitan monooleate, (4) condensation products of said partial esters with ethylene oxide, for example, polyoxyethylene sorbitan monooleate. The emulsions may also contain sweetening and flavoring agents.

Oily suspensions may be formulated by suspending the active ingredient in a vegetable oil such as, for example, arachis oil, olive oil, sesame oil or coconut oil, or in a mineral oil such as liquid paraffin. The oily suspensions may contain a thickening agent such as, for example, beeswax, hard paraffin, or cetyl alcohol. The suspensions may also contain one or more preservatives, for example, ethyl or n-propyl p-hydroxybenzoate; one or more coloring agents; one or more flavoring agents; and one or more sweetening agents such as sucrose or saccharin.

Syrups and elixirs may be formulated with sweetening agents such as, for example, glycerol, propylene glycol, sorbitol or sucrose. Such formulations may also contain a demulcent, and preservative, such as methyl and propyl parabens and flavoring and coloring agents.

The compounds of this invention may also be administered parenterally, that is, subcutaneously, intravenously, intraocularly, intrasynovially, intramuscularly, or interperitoneally, as injectable dosages of the compound in preferably a physiologically acceptable diluent with a pharmaceutical carrier which can be a sterile liquid or mixture of liquids such as water, saline, aqueous dextrose and related sugar solutions, an alcohol such as ethanol, isopropanol, or hexadecyl alcohol, glycols such as propylene glycol or polyethylene glycol, glycerol ketals such as 2,2-dimethyl-1,1-dioxolane-4-methanol, ethers such as poly(ethylene glycol) 400, an oil, a fatty acid, a fatty acid ester or, a fatty acid glyceride, or an acetylated fatty acid glyceride, with or without the addition of a pharmaceutically acceptable surfactant such as a soap or a detergent, suspending agent such as pectin, carbomers, methycellulose, hydroxypropylmethylcellulose, or carboxymethylcellulose, or emulsifying agent and other pharmaceutical adjuvants.

Illustrative of oils which can be used in the parenteral formulations of this invention are those of petroleum, animal, vegetable, or synthetic origin, for example, peanut oil, soybean oil, sesame oil, cottonseed oil, corn oil, olive oil, petrolatum and mineral oil. Suitable fatty acids include oleic acid, stearic acid, isostearic acid and myristic acid. Suitable fatty acid esters are, for example, ethyl oleate and isopropyl myristate. Suitable soaps include fatty acid alkali metal, ammonium, and triethanolamine salts and suitable detergents include cationic detergents, for example dimethyl dialkyl ammonium halides, alkyl pyridinium halides, and alkylamine acetates; anionic detergents, for example, alkyl, aryl, and olefin sulfonates, alkyl, olefin, ether, and monoglyceride sulfates, and sulfosuccinates; non-ionic detergents, for example, fatty amine oxides, fatty acid alkanolamides, and poly(oxyethylene-oxypropylene)s or ethylene oxide or propylene oxide copolymers; and amphoteric detergents, for example, alkyl-beta-aminopropionates, and 2-alkylimidazoline quarternary ammonium salts, as well as mixtures.

The parenteral compositions of this invention will typically contain from about 0.5% to about 25% by weight of the active ingredient in solution. Preservatives and buffers may also be used advantageously. In order to minimize or eliminate irritation at the site of injection, such compositions may contain a non-ionic surfactant having a hydrophile-lipophile balance (HLB) preferably of from about 12 to about 17. The quantity of surfactant in such formulation preferably ranges from about 5% to about 15% by weight. The surfactant can be a single component having the above HLB or can be a mixture of two or more components having the desired HLB.

Illustrative of surfactants used in parenteral formulations are the class of polyethylene sorbitan fatty acid esters, for example, sorbitan monooleate and the high molecular weight adducts of ethylene oxide with a hydrophobic base, formed by the condensation of propylene oxide with propylene glycol.

The pharmaceutical compositions may be in the form of sterile injectable aqueous suspensions. Such suspensions may be formulated according to known methods using suitable dispersing or wetting agents and suspending agents such as, for example, sodium carboxymethylcellulose, methylcellulose, hydroxypropylmethyl-cellulose, sodium alginate, polyvinylpyrrolidone, gum tragacanth and gum acacia; dispersing or wetting agents which may be a naturally occurring phosphatide such as lecithin, a condensation product of an alkylene oxide with a fatty acid, for example, polyoxyethylene stearate, a condensation product of ethylene oxide with a long chain aliphatic alcohol, for example, heptadeca-ethyleneoxycetanol, a condensation product of ethylene oxide with a partial ester derived form a fatty acid and a hexitol such as polyoxyethylene sorbitol monooleate, or a condensation product of an ethylene oxide with a partial ester derived from a fatty acid and a hexitol anhydride, for example polyoxyethylene sorbitan monooleate.

The sterile injectable preparation may also be a sterile injectable solution or suspension in a non-toxic parenterally acceptable diluent or solvent. Diluents and solvents that may be employed are, for example, water, Ringer's solution, isotonic sodium chloride solutions and isotonic glucose solutions. In addition, sterile fixed oils are conventionally employed as solvents or suspending media. For this purpose, any bland, fixed oil may be employed including synthetic mono- or diglycerides. In addition, fatty acids such as oleic acid can be used in the preparation of injectables.

A composition of the invention may also be administered in the form of suppositories for rectal administration of the drug. These compositions can be prepared by mixing the drug with a suitable non-irritation excipient which is solid at ordinary temperatures but liquid at the rectal temperature and will therefore melt in the rectum to release the drug. Such materials are, for example, cocoa butter and polyethylene glycol.

Another formulation employed in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art (see, e.g., U.S. Pat. No. 5,023,252, issued Jun. 11, 1991, incorporated herein by reference). Such patches may be constructed for continuous, pulsatile, or on demand delivery of pharmaceutical agents.

Controlled release formulations for parenteral administration include liposomal, polymeric microsphere and polymeric gel formulations that are known in the art.

It may be desirable or necessary to introduce the pharmaceutical composition to the patient via a mechanical delivery device. The construction and use of mechanical delivery devices for the delivery of pharmaceutical agents is well known in the art. Direct techniques for, for example, administering a drug directly to the brain usually involve placement of a drug delivery catheter into the patient's ventricular system to bypass the blood-brain barrier. One such implantable delivery system, used for the transport of agents to specific anatomical regions of the body, is described in U.S. Pat. No. 5,011,472, issued Apr. 30, 1991.

The compositions of the invention can also contain other conventional pharmaceutically acceptable compounding ingredients, generally referred to as carriers or diluents, as necessary or desired. Conventional procedures for preparing such compositions in appropriate dosage forms can be utilized. Such ingredients and procedures include those described in the following references, each of which is incorporated herein by reference: Powell, M. F. et al, “Compendium of Excipients for Parenteral Formulations” PDA Journal of Pharmaceutical Science & Technology 1998, 52(5), 238-311; Strickley, R. G “Parenteral Formulations of Small Molecule Therapeutics Marketed in the United States (1999)-Part-1” PDA Journal of Pharmaceutical Science & Technology 1999, 53(6), 324-349; and Nema, S. et al, “Excipients and Their Use in Injectable Products” PDA Journal of Pharmaceutical Science & Technology 1997, 51(4), 166-171.

Commonly used pharmaceutical ingredients that can be used as appropriate to formulate the composition for its intended route of administration include:

acidifying agents (examples include but are not limited to acetic acid, citric acid, fumaric acid, hydrochloric acid, nitric acid);

alkalinizing agents (examples include but are not limited to ammonia solution, ammonium carbonate, diethanolamine, monoethanolamine, potassium hydroxide, sodium borate, sodium carbonate, sodium hydroxide, triethanolamine, trolamine);

adsorbents (examples include but are not limited to powdered cellulose and activated charcoal);

aerosol propellants (examples include but are not limited to carbon dioxide, CC₂F₂, F₂ClC—CClF₂ and CClF₃)

air displacement agents (examples include but are not limited to nitrogen and argon);

antifungal preservatives (examples include but are not limited to benzoic acid, butylparaben, ethylparaben, methylparaben, propylparaben, sodium benzoate);

antimicrobial preservatives (examples include but are not limited to benzalkonium chloride, benzethonium chloride, benzyl alcohol, cetylpyridinium chloride, chlorobutanol, phenol, phenylethyl alcohol, phenylmercuric nitrate and thimerosal);

antioxidants (examples include but are not limited to ascorbic acid, ascorbyl palmitate, butylated hydroxyanisole, butylated hydroxytoluene, hypophosphorus acid, monothioglycerol, propyl gallate, sodium ascorbate, sodium bisulfite, sodium formaldehyde sulfoxylate, sodium metabisulfite);

binding materials (examples include but are not limited to block polymers, natural and synthetic rubber, polyacrylates, polyurethanes, silicones, polysiloxanes and styrene-butadiene copolymers);

buffering agents (examples include but are not limited to potassium metaphosphate, dipotassium phosphate, sodium acetate, sodium citrate anhydrous and sodium citrate dihydrate)

carrying agents (examples include but are not limited to acacia syrup, aromatic syrup, aromatic elixir, cherry syrup, cocoa syrup, orange syrup, syrup, corn oil, mineral oil, peanut oil, sesame oil, bacteriostatic sodium chloride injection and bacteriostatic water for injection)

chelating agents (examples include but are not limited to edetate disodium and edetic acid)

colorants (examples include but are not limited to FD&C Red No. 3, FD&C Red No. 20, FD&C Yellow No. 6, FD&C Blue No. 2, D&C Green No. 5, D&C Orange No. 5, D&C Red No. 8, caramel and ferric oxide red);

clarifying agents (examples include but are not limited to bentonite);

emulsifying agents (examples include but are not limited to acacia, cetomacrogol, cetyl alcohol, glyceryl monostearate, lecithin, sorbitan monooleate, polyoxyethylene 50 monostearate);

encapsulating agents (examples include but are not limited to gelatin and cellulose acetate phthalate)

flavorants (examples include but are not limited to anise oil, cinnamon oil, cocoa, menthol, orange oil, peppermint oil and vanillin);

humectants (examples include but are not limited to glycerol, propylene glycol and sorbitol);

levigating agents (examples include but are not limited to mineral oil and glycerin);

oils (examples include but are not limited to arachis oil, mineral oil, olive oil, peanut oil, sesame oil and vegetable oil);

ointment bases (examples include but are not limited to lanolin, hydrophilic ointment, polyethylene glycol ointment, petrolatum, hydrophilic petrolatum, white ointment, yellow ointment, and rose water ointment);

penetration enhancers (transdermal delivery) (examples include but are not limited to monohydroxy or polyhydroxy alcohols, mono- or polyvalent alcohols, saturated or unsaturated fatty alcohols, saturated or unsaturated fatty esters, saturated or unsaturated dicarboxylic acids, essential oils, phosphatidyl derivatives, cephalin, terpenes, amides, ethers, ketones and ureas)

plasticizers (examples include but are not limited to diethyl phthalate and glycerol);

solvents (examples include but are not limited to ethanol, corn oil, cottonseed oil, glycerol, isopropanol, mineral oil, oleic acid, peanut oil, purified water, water for injection, sterile water for injection and sterile water for irrigation);

stiffening agents (examples include but are not limited to cetyl alcohol, cetyl esters wax, microcrystalline wax, paraffin, stearyl alcohol, white wax and yellow wax);

suppository bases (examples include but are not limited to cocoa butter and polyethylene glycols (mixtures));

surfactants (examples include but are not limited to benzalkonium chloride, nonoxynol 10, oxtoxynol 9, polysorbate 80, sodium lauryl sulfate and sorbitan mono-palmitate);

suspending agents (examples include but are not limited to agar, bentonite, carbomers, carboxymethylcellulose sodium, hydroxyethyl cellulose, hydroxypropyl cellulose, hydroxypropyl methylcellulose, kaolin, methylcellulose, tragacanth and veegum);

sweetening agents (examples include but are not limited to aspartame, dextrose, glycerol, mannitol, propylene glycol, saccharin sodium, sorbitol and sucrose);

tablet anti-adherents (examples include but are not limited to magnesium stearate and talc);

tablet binders (examples include but are not limited to acacia, alginic acid, carboxymethylcellulose sodium, compressible sugar, ethylcellulose, gelatin, liquid glucose, methylcellulose, non-crosslinked polyvinyl pyrrolidone, and pregelatinized starch);

tablet and capsule diluents (examples include but are not limited to dibasic calcium phosphate, kaolin, lactose, mannitol, microcrystalline cellulose, powdered cellulose, precipitated calcium carbonate, sodium carbonate, sodium phosphate, sorbitol and starch);

tablet coating agents (examples include but are not limited to liquid glucose, hydroxyethyl cellulose, hydroxypropyl cellulose, hydroxypropyl methylcellulose, methylcellulose, ethylcellulose, cellulose acetate phthalate and shellac);

tablet direct compression excipients (examples include but are not limited to dibasic calcium phosphate);

tablet disintegrants (examples include but are not limited to alginic acid, carboxymethylcellulose calcium, microcrystalline cellulose, polacrillin potassium, crosslinked polyvinylpyrrolidone, sodium alginate, sodium starch glycollate and starch);

tablet glidants (examples include but are not limited to colloidal silica, corn starch and talc);

tablet lubricants (examples include but are not limited to calcium stearate, magnesium stearate, mineral oil, stearic acid and zinc stearate);

tablet/capsule opaquants (examples include but are not limited to titanium dioxide);

tablet polishing agents (examples include but are not limited to carnuba wax and white wax);

thickening agents (examples include but are not limited to beeswax, cetyl alcohol and paraffin);

tonicity agents (examples include but are not limited to dextrose and sodium chloride);

viscosity increasing agents (examples include but are not limited to alginic acid, bentonite, carbomers, carboxymethylcellulose sodium, methylcellulose, polyvinyl pyrrolidone, sodium alginate and tragacanth); and

wetting agents (examples include but are not limited to heptadecaethylene oxycetanol, lecithins, sorbitol monooleate, polyoxyethylene sorbitol monooleate, and polyoxyethylene stearate).

Based upon standard laboratory techniques known to evaluate compounds and compositions useful for the methods of the present invention, by standard toxicity tests and by standard pharmacological assays for the determination of treatment of the conditions identified above in mammals, and by comparison of these results with the results of known medicaments that are used to treat these conditions, the effective dosage of the compounds of this invention can readily be determined for treatment of each desired indication. The amount of the active ingredients to be administered in the treatment of one of these conditions can vary widely according to such considerations as the particular compound and dosage unit employed, the mode of administration, the period of treatment, the age and sex of the patient treated, and the nature and extent of the condition treated.

The total amount of the active ingredients to be administered will generally range from about 0.001 mg/kg to about 200 mg/kg body weight per day, and preferably from about 0.01 mg/kg to about 20 mg/kg body weight per day. Clinically useful dosing schedules will range from one to three times a day dosing to once every four weeks dosing. In addition, “drug holidays” in which a patient is not dosed with a drug for a certain period of time, may be beneficial to the overall balance between pharmacological effect and tolerability. A unit dosage may contain from about 0.5 mg to about 1500 mg of active ingredient, and can be administered one or more times per day or less than once a day. The average daily dosage for administration by injection, including intravenous, intramuscular, subcutaneous and parenteral injections, and use of infusion techniques will preferably be from 0.01 to 200 mg/kg of total body weight. The average daily rectal dosage regimen will preferably be from 0.01 to 200 mg/kg of total body weight. The average daily vaginal dosage regimen will preferably be from 0.01 to 200 mg/kg of total body weight. The average daily topical dosage regimen will preferably be from 0.1 to 200 mg administered between one to four times daily. The transdermal concentration will preferably be that required to maintain a daily dose of from 0.01 to 200 mg/kg. The average daily inhalation dosage regimen will preferably be from 0.01 to 100 mg/kg of total body weight.

Of course the specific initial and continuing dosage regimen for each patient will vary according to the nature and severity of the condition as determined by the attending diagnostician, the activity of the specific compound employed, the age and general condition of the patient, time of administration, route of administration, rate of excretion of the drug, drug combinations, and the like. The desired mode of treatment and number of doses of a compound of the present invention or a pharmaceutically acceptable salt or ester or composition thereof can be ascertained by those skilled in the art using conventional treatment tests.

In a further aspect, there is provided a kit for use in a method according to the invention. Preferably such a kit comprises all the components necessary to practice a method as described herein.

In another aspect, the description provides a method of treating or preventing a disease, comprising performing a method as described herein and administering a therapeutic agent as described herein either alone or in combination with an effective amount of another additional therapeutic or bioactive agent, e.g., antibiotic, anti-cancer agent, anti-inflammatory, antimicrobial, antiviral, antifungal, antipsychotic, etc. The term “bioactive agent” is used to describe an agent with biological activity to assist in effecting an intended therapy, inhibition and/or prevention/prophylaxis. The terms “treat”, “treating”, and “treatment”, etc., as used herein, refer to any action providing a benefit to a patient including the treatment of any disease state or condition. The additional therapeutic or bioactive agent may be administered concurrently or sequentially with the composition of the invention.

Disease states of conditions which may be treated using compounds according to the present invention include, for example, asthma, autoimmune diseases such as multiple sclerosis, various cancers, ciliopathies, cleft palate, diabetes, heart disease, hypertension, inflammatory bowel disease, mental retardation, mood disorder, obesity, refractive error, infertility, Angelman syndrome, Canavan disease, Coeliac disease, Charcot-Marie-Tooth disease, Cystic fibrosis, Duchenne muscular dystrophy, Haemochromatosis, Hemophilia, Klinefelter's syndrome, Neurofibromatosis, Phenylketonuria, Polycystic kidney disease, (PKD1) or 4 (PKD2) Prader-Willi syndrome, Sickle-cell disease, Tay-Sachs disease, Turner syndrome.

Further disease states or conditions which may be treated by compounds according to the present invention include Alzheimer's disease, Amyotrophic lateral sclerosis (Lou Gehrig's disease), Anorexia nervosa, Anxiety disorder, Atherosclerosis, Attention deficit hyperactivity disorder, Autism, Bipolar disorder, Chronic fatigue syndrome, Chronic obstructive pulmonary disease, Crohn's disease, Coronary heart disease, Dementia, Depression, Diabetes mellitus type 1, Diabetes mellitus type 2, Epilepsy, Guillain-Barré syndrome, Irritable bowel syndrome, Lupus, Metabolic syndrome, Multiple sclerosis, Myocardial infarction, Obesity, Obsessive-compulsive disorder, Panic disorder, Parkinson's disease, Psoriasis, Rheumatoid arthritis, Sarcoidosis, Schizophrenia, Stroke, Thromboangiitis obliterans, Tourette syndrome, Vasculitis.

Still additional disease states or conditions which can be treated by compounds according to the present invention include aceruloplasminemia, Achondrogenesis type II, achondroplasia, Acrocephaly, Gaucher disease type 2, acute intermittent porphyria, Canavan disease, Adenomatous Polyposis Coli, ALA dehydratase deficiency, adenylosuccinate lyase deficiency, Adrenogenital syndrome, Adrenoleukodystrophy, ALA-D porphyria, ALA dehydratase deficiency, Alkaptonuria, Alexander disease, Alkaptonuric ochronosis, alpha 1-antitrypsin deficiency, alpha-1 proteinase inhibitor, emphysema, amyotrophic lateral sclerosis Alstram syndrome, Alexander disease, Amelogenesis imperfecta, ALA dehydratase deficiency, Anderson-Fabry disease, androgen insensitivity syndrome, Anemia Angiokeratoma Corporis Diffusum, Angiomatosis retinae (von Hippel-Lindau disease) Apert syndrome, Arachnodactyly (Marfan syndrome), Stickler syndrome, Arthrochalasis multiplex congenital (Ehlers-Danlos syndrome#arthrochalasia type) ataxia telangiectasia, Rett syndrome, primary pulmonary hypertension, Sandhoff disease, neurofibromatosis type II, Beare-Stevenson cutis gyrata syndrome, Mediterranean fever, familial, Benjamin syndrome, beta-thalassemia, Bilateral Acoustic Neurofibromatosis (neurofibromatosis type II), factor V Leiden thrombophilia, Bloch-Sulzberger syndrome (incontinentia pigmenti), Bloom syndrome, X-linked sideroblastic anemia, Bonnevie-Ullrich syndrome (Turner syndrome), Bourneville disease (tuberous sclerosis), prion disease, Birt-Hogg-Dub6 syndrome, Brittle bone disease (osteogenesis imperfecta), Broad Thumb-Hallux syndrome (Rubinstein-Taybi syndrome), Bronze Diabetes/Bronzed Cirrhosis (hemochromatosis), Bulbospinal muscular atrophy (Kennedy's disease), Burger-Grutz syndrome (lipoprotein lipase deficiency), CGD Chronic granulomatous disorder, Campomelic dysplasia, biotinidase deficiency, Cardiomyopathy (Noonan syndrome), Cri du chat, CAVD (congenital absence of the vas deferens), Caylor cardiofacial syndrome (CBAVD), CEP (congenital erythropoietic porphyria), cystic fibrosis, congenital hypothyroidism, Chondrodystrophy syndrome(achondroplasia), otospondylomegaepiphyseal dysplasia, Lesch-Nyhan syndrome, galactosemia, Ehlers-Danlos syndrome, Thanatophoric dysplasia, Coffin-Lowry syndrome, Cockayne syndrome, (familial adenomatous polyposis), Congenital erythropoietic porphyria, Congenital heart disease, Methemoglobinemia/Congenital methaemoglobinaemia, achondroplasia, X-linked sideroblastic anemia, Connective tissue disease, Conotruncal anomaly face syndrome, Cooley's Anemia (beta-thalassemia), Copper storage disease (Wilson's disease), Copper transport disease (Menkes disease), hereditary coproporphyria, Cowden syndrome, Craniofacial dysarthrosis (Crouzon syndrome), Creutzfeldt-Jakob disease (prion disease), Cockayne syndrome, Cowden syndrome, Curschmann-Batten-Steinert syndrome (myotonic dystrophy), Beare-Stevenson cutis gyrata syndrome, primary hyperoxaluria, spondyloepimetaphyseal dysplasia (Strudwick type), muscular dystrophy, Duchenne and Becker types (DBMD), Usher syndrome, Degenerative nerve diseases including de Grouchy syndrome and Dejerine-Sottas syndrome, developmental disabilities, distal spinal muscular atrophy, type V, androgen insensitivity syndrome, Diffuse Globoid Body Sclerosis (Krabbe disease), Di George's syndrome, Dihydrotestosterone receptor deficiency, androgen insensitivity syndrome, Down syndrome, Dwarfism, erythropoietic protoporphyria Erythroid 5-aminolevulinate synthetase deficiency, Erythropoietic porphyria, erythropoietic protoporphyria, erythropoietic uroporphyria, Friedreich's ataxia, familial paroxysmal polyserositis, porphyria cutanea tarda, familial pressure sensitive neuropathy, primary pulmonary hypertension (PPH), Fibrocystic disease of the pancreas, fragile X syndrome, galactosemia, genetic brain disorders, Giant cell hepatitis (Neonatal hemochromatosis), Gronblad-Strandberg syndrome (pseudoxanthoma elasticum), Gunther disease (congenital erythropoietic porphyria), haemochromatosis, Hallgren syndrome, sickle cell anemia, hemophilia, hepatoerythropoietic porphyria (HEP), Hippel-Lindau disease (von Hippel-Lindau disease), Huntington's disease, Hutchinson-Gilford progeria syndrome (progeria), Hyperandrogenism, Hypochondroplasia, Hypochromic anemia, Immune system disorders, including X-linked severe combined immunodeficiency, Insley-Astley syndrome, Jackson-Weiss syndrome, Joubert syndrome, Lesch-Nyhan syndrome, Jackson-Weiss syndrome, Kidney diseases, including hyperoxaluria, Klinefelter's syndrome, Kniest dysplasia, Lacunar dementia, Langer-Saldino achondrogenesis, ataxia telangiectasia, Lynch syndrome, Lysyl-hydroxylase deficiency, Machado-Joseph disease, Metabolic disorders, including Kniest dysplasia, Marfan syndrome, Movement disorders, Mowat-Wilson syndrome, cystic fibrosis, Muenke syndrome, Multiple neurofibromatosis, Nance-Insley syndrome, Nance-Sweeney chondrodysplasia, Niemann-Pick disease, Noack syndrome (Pfeiffer syndrome), Osler-Weber-Rendu disease, Peutz-Jeghers syndrome, Polycystic kidney disease, polyostotic fibrous dysplasia (McCune-Albright syndrome), Peutz-Jeghers syndrome, Prader-Labhart-Willi syndrome, hemochromatosis, primary hyperuricemia syndrome (Lesch-Nyhan syndrome), primary pulmonary hypertension, primary senile degenerative dementia, prion disease, progeria (Hutchinson Gilford Progeria Syndrome), progressive chorea, chronic hereditary (Huntington) (Huntington's disease), progressive muscular atrophy, spinal muscular atrophy, propionic acidemia, protoporphyria, proximal myotonic dystrophy, pulmonary arterial hypertension, PXE (pseudoxanthoma elasticum), Rb (retinoblastoma), Recklinghausen disease (neurofibromatosis type I), Recurrent polyserositis, Retinal disorders, Retinoblastoma, Rett syndrome, RFALS type 3, Ricker syndrome, Riley-Day syndrome, Roussy-Levy syndrome, severe achondroplasia with developmental delay and acanthosis nigricans (SADDAN), Li-Fraumeni syndrome, sarcoma, breast, leukemia, and adrenal gland (SBLA) syndrome, sclerosis tuberose (tuberous sclerosis), SDAT, SED congenital (spondyloepiphyseal dysplasia congenita), SED Strudwick (spondyloepimetaphyseal dysplasia, Strudwick type), SEDc (spondyloepiphyseal dysplasia congenita) SEMD, Strudwick type (spondyloepimetaphyseal dysplasia, Strudwick type), Shprintzen syndrome, Skin pigmentation disorders, Smith-Lemli-Opitz syndrome, South-African genetic porphyria (variegate porphyria), infantile-onset ascending hereditary spastic paralysis, Speech and communication disorders, sphingolipidosis, Tay-Sachs disease, spinocerebellar ataxia, Stickler syndrome, stroke, androgen insensitivity syndrome, tetrahydrobiopterin deficiency, beta-thalassemia, Thyroid disease, Tomaculous neuropathy (hereditary neuropathy with liability to pressure palsies), Treacher Collins syndrome, Triplo X syndrome (triple X syndrome), Trisomy 21 (Down syndrome), Trisomy X, VHL syndrome (von Hippel-Lindau disease), Vision impairment and blindness (Alström syndrome), Vrolik disease, Waardenburg syndrome, Warburg Sjo Fledelius Syndrome, Weissenbacher-Zweymüller syndrome, Wolf-Hirschhorn syndrome, Wolff Periodic disease, Weissenbacher-Zweymüller syndrome and Xeroderma pigmentosum, among others.

The term “cancer” refers to the pathological process that results in the formation and growth of a cancerous or malignant neoplasm, i.e., abnormal tissue that grows by cellular proliferation, often more rapidly than normal and continues to grow after the stimuli that initiated the new growth cease. Malignant neoplasms show partial or complete lack of structural organization and functional coordination with the normal tissue and most invade surrounding tissues, metastasize to several sites, and are likely to recur after attempted removal and to cause the death of the patient unless adequately treated. Exemplary cancers which may be treated by the present compounds either alone or in combination with at least one additional anti-cancer agent include squamous-cell carcinoma, basal cell carcinoma, adenocarcinoma, hepatocellular carcinomas, and renal cell carcinomas, cancer of the bladder, bowel, breast, cervix, colon, esophagus, head, kidney, liver, lung, neck, ovary, pancreas, prostate, and stomach; leukemias; benign and malignant lymphomas, particularly Burkitt's lymphoma and Non-Hodgkin's lymphoma; benign and malignant melanomas; myeloproliferative diseases; sarcomas, including Ewing's sarcoma, hemangiosarcoma, Kaposi's sarcoma, liposarcoma, myosarcomas, peripheral neuroepithelioma, synovial sarcoma, gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas, and Schwannomas; bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, melanoma; carcinosarcoma, Hodgkin's disease, Wilms' tumor and teratocarcinomas. Additional cancers which may be treated using compounds according to the present invention include, for example, T-lineage Acute lymphoblastic Leukemia (T-ALL), T-lineage lymphoblastic Lymphoma (T-LL), Peripheral T-cell lymphoma, Adult T-cell Leukemia, Pre-B ALL, Pre-B Lymphomas, Large B-cell Lymphoma, Burkitts Lymphoma, B-cell ALL, Philadelphia chromosome positive ALL and Philadelphia chromosome positive CML.

The term “anti-cancer agent” is used to describe an anti-cancer agent. These agents include, for example, everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, a FLT-3 inhibitor, a VEGFR inhibitor, an EGFR TK inhibitor, an aurora kinase inhibitor, a PIK-1 modulator, a Bcl-2 inhibitor, an HDAC inhbitor, a c-MET inhibitor, a PARP inhibitor, a Cdk inhibitor, an EGFR TK inhibitor, an IGFR-TK inhibitor, an anti-HGF antibody, a PI3 kinase inhibitor, an AKT inhibitor, an mTORCI/2 inhibitor, a JAK/STAT inhibitor, a checkpoint-1 or 2 inhibitor, a focal adhesion kinase inhibitor, a Map kinase (mek) inhibitor, a VEGF trap antibody, pemetrexed, erlotinib, dasatanib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdR KRX-0402, lucanthone, LY317615, neuradiab, vitespan, Rta 744, Sdx 102, talampanel, atrasentan, Xr 311, romidepsin, ADS-100380, sunitinib, 5-fluorouracil, vorinostat, etoposide, gemcitabine, doxorubicin, liposomal doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1H-pyrrolo[2,3-d]pyrimidin-5-yl)ethyl]benzoyl]-, disodium salt, heptahydrate, camptothecin, PEG-labeled irinotecan, tamoxifen, toremifene citrate, anastrazole, exemestane, letrozole, DES(diethylstilbestrol), estradiol, estrogen, conjugated estrogen, bevacizumab, IMC-1C11, CHIR-258); 3-[5-(methylsulfonylpiperadinemethyl)-indolyl-quinolone, vatalanib, AG-013736, AVE-0005, goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, Ionafarnib, BMS-214662, tipifarnib; amifostine, NVP-LAQ824, suberoyl analide hydroxamic acid, valproic acid, trichostatin A, FK-228, SU11248, sorafenib, KRN951, aminoglutethimide, arnsacrine, anagrelide, L-asparaginase, Bacillus Calmette-Guerin (BCG) vaccine, adriamycin, bleomycin, buserelin, busulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, gleevec, gemcitabine, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mecaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezimib, paclitaxel, cremophor-free paclitaxel, docetaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, topotecan, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonist, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa, darbepoetin alfa and mixtures thereof.

The term “antivirals” include, for example, nucleoside reverse transcriptase inhibitors (NRTI), other non-nucleoside reverse transcriptase inhibitors (i.e., those which are not representative of the present invention), protease inhibitors, fusion inhibitors, among others, exemplary compounds of which may include, for example, 3TC (Lamivudine), AZT (Zidovudine), (−)-FTC, ddI (Didanosine), ddC (zalcitabine), abacavir (ABC), tenofovir (PMPA), D-D4FC (Reverset), D4T (Stavudine), Racivir, L-FddC, L-FD4C, NVP (Nevirapine), DLV (Delavirdine), EFV (Efavirenz), SQVM (Saquinavir mesylate), RTV (Ritonavir), IDV (Indinavir), SQV (Saquinavir), NFV (Nelfinavir), APV (Amprenavir), LPV (Lopinavir), fusion inhibitors such as T20, among others, fuseon and mixtures thereof, including anti-HIV compounds presently in clinical trials or in development.

Other anti-HIV agents which may be used include, for example, other NNRTI's (i.e., other than the NNRTI's according to the present invention) may be selected from the group consisting of nevirapine (BI-R6-587), delavirdine (U-901525/T), efavirenz (DMP-266), UC-781 (N-[4-chloro-3-(3-methyl-2-butenyloxy)phenyl]-2methyl3-furancarbothiamide), etravirine (TMC125), Trovirdine (Ly300046.HCl), MKC-442 (emivirine, coactinon), HI-236, HI-240, HI-280, HI-281, rilpivirine (TMC-278), MSC-127, HBY 097, DMP266, Baicalin (TJN-151) ADAM-II (Methyl 3′,3′-dichloro-4′,4″-dimethoxy-5′,5″-bis(methoxycarbonyl)-6,6-diphenylhexenoate), Methyl 3-Bromo-5-(1-5-bromo-4-methoxy-3-(methoxycarbonyl)phenyl)hept-1-enyl)-2-methoxybenzoate (Alkenyldiarylmethane analog, Adam analog), (5-chloro-3-(phenylsulfinyl)-2′-indolecarboxamide), AAP-BHAP (U-104489 or PNU-104489), Capravirine (AG-1549, S-1153), atevirdine (U-87201E), aurin tricarboxylic acid (SD-095345), 1-[(6-cyano-2-indolyl)carbonyl]-4-[3-(isopropylamino)-2-pyridinyl]piperazine, 1-[5-[[N-(methyl)methylsulfonylamino]-2-indolylcarbonyl-4-[3-(isopropylamino)-2-pyridinyl]piperazine, 1-[3-(Ethylamino)-2-[pyridinyl]-4-[(5-hydroxy-2-indolyl)carbonyl]piperazine, 1-[(6-Formyl-2-indolyl)carbonyl]-4-[3-(isopropylamino)-2-pyridinyl]piperazine, 1-[[5-(Methylsulfonyloxy)-2-indoyly)carbonyl]-4-[3-(isopropylamino)-2-pyridinyl]piperazine, U88204E, Bis(2-nitrophenyl)sulfone (NSC 633001), Calanolide A (NSC675451), Calanolide B, 6-Benzyl-5-methyl-2-(cyclohexyloxy)pyrimidin-4-one (DABO-546), DPC 961, E-EBU, E-EBU-dm, E-EPSeU, E-EPU, Foscarnet (Foscavir), HEPT (1-[(2-Hydroxyethoxy)methyl]-6-(phenylthio)thymine), HEPT-M (1-[(2-Hydroxyethoxy)methyl]-6-(3-methylphenyl)thio)thymine), HEPT-S(1-[(2-Hydroxyethoxy)methyl]-6-(phenylthio)-2-thiothymine), Inophyllum P, L-737,126, Michellamine A (NSC650898), Michellamine B (NSC649324), Michellamine F, 6-(3,5-Dimethylbenzyl)-1-[(2-hydroxyethoxy)methyl]-5-isopropyluracil, 6-(3,5-Dimethylbenzyl)-1-(ethyoxymethyl)-5-isopropyluracil, NPPS, E-BPTU (NSC 648400), Oltipraz (4-Methyl-5-(pyrazinyl)-3H-1,2-dithiole-3-thione), N-{2-(2-Chloro-6-fluorophenethyl]-N′-(2-thiazolyl)thiourea (PETT Cl, F derivative), N-{2-(2,6-Difluorophenethyl]-N′-[2-(5-bromopyridyl)]thiourea {PETT derivative), N-{2-(2,6-Difluorophenethyl]-N′-[2-(5-methylpyridyl)]thiourea {PETT Pyridyl derivative), N-[2-(3-Fluorofuranyl)ethyl]-N′-[2-(5-chloropyridyl)]thiourea, N-[2-(2-Fluoro-6-ethoxyphenethyl)]-N′-[2-(5-bromopyridyl)]thiourea, N-(2-Phenethyl)-N′-(2-thiazolyl)thiourea (LY-73497), L-697,639, L-697,593, L-697,661, 3-[2-(4,7-Difluorobenzoxazol-2-yl)ethyl}-5-ethyl-6-methyl(pypridin-2(1H)-thione (2-Pyridinone Derivative), 3-[[(2-Methoxy-5,6-dimethyl-3-pyridyl)methyl]amine]-5-ethyl-6-methyl(pypridin-2(1H)-thione, R82150, R82913, R87232, R88703, R89439 (Loviride), R90385, S-2720, Suramin Sodium, TBZ (Thiazolobenzimidazole, NSC 625487), Thiazoloisoindol-5-one, (+)(R)-9b-(3,5-Dimethylphenyl-2,3-dihydrothiazolo [2,3-a]isoindol-5(9bH)-one, Tivirapine (R86183), UC-38 and UC-84, among others.

Antimicrobial agents include, e.g., antibiotics. In certain embodiments, the anti-microbial is an anti-tuberculosis drug, e.g., pyrazinamide or benzamide, pretomanid, and bedaquiline, among others.

### EXAMPLES

The contents of all references, patents, pending patent applications and published patents, cited throughout this application are hereby expressly incorporated by reference. The above described compositions and methods are further exemplified by reference to the Figures and accompanying description below.

PPi-Dependent Reverse Reaction Generates dNTP

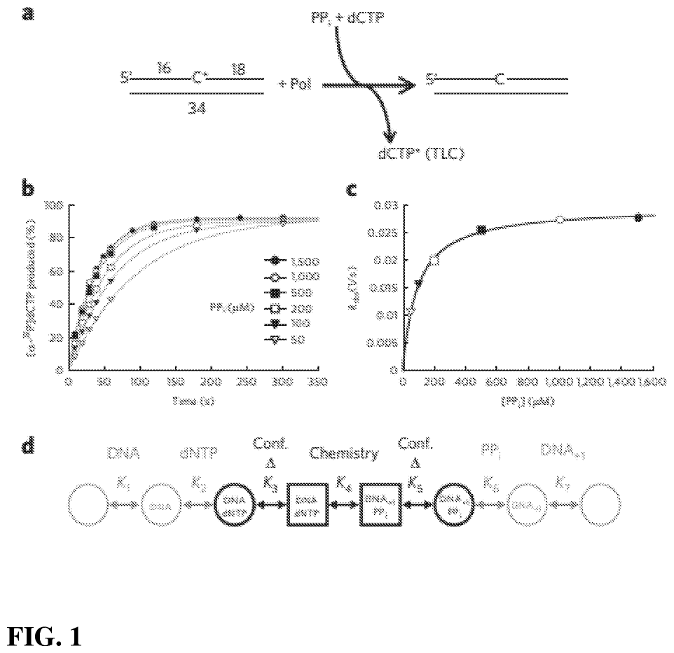

Because nicked DNA positions the primer terminus at the N site, a nicked substrate with a ³²P-labeled 3′ primer terminus was routinely used for kinetic measurements (FIG. 1a). With this DNA substrate, pyrophosphorolysis generates [α-32P] dNTP and single nucleotide gapped DNA. Pyrophosphorolysis can be observed by the loss of radioactively labeled DNA or by the formation of radiolabeled dNTP. Following the formation of [32P] dCTP using thin-layer chromatography (TLC), the observed rate of the single-turnover (enzyme/DNA>1) time courses was shown to be dependent on PPi concentration (FIG. 1b). A secondary plot of these observed rate constants provided the apparent PPi binding affinity (˜90 μM) and observed rate constant for pyrophosphorolysis (krev ˜0.03 s⁻¹) (FIG. 1c; Supplementary Results, Supplementary Table 1).

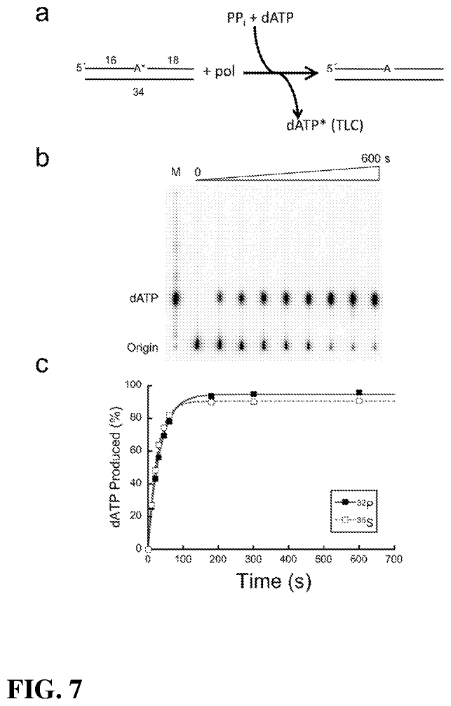

As ligand (dNTP or PPi) binding results in a structural transition from an open to closed polymerase conformation that is necessary for catalysis, large global conformational changes occur before and after chemistry (FIG. 1d). To ascertain whether the slow rate of the reverse reaction is limited by chemistry or by a nonchemical step, the rate of removal of a 3′-terminal phosphorothioate was determined. Due to different steric, electronic, and metal-binding characteristics of sulfur relative to oxygen, a substantial decrease in rate upon sulfur substitution would suggest that chemistry is rate limiting. The substrate was enzymatically synthesized using the Sp-diastereomer of (u-S)dATP. Because there is inversion of configuration, the reaction generates nicked DNA with a 3′-terminal Rp-phosphorothioate internucleotide linkage. In contrast to the forward reaction, there is no phosphorothioate elemental effect observed for pyrophosphorolysis (FIG. 7), indicating that chemistry is not rate limiting. In addition, the rate constant for pyrophosphorolysis with T-A in the nick was similar to that measured with G-C.

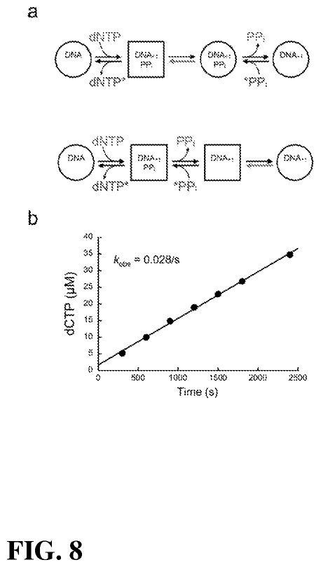

To determine whether PPi binding could circumvent a kinetic roadblock, an exchange reaction was measured to follow the movement of radioactive label in [³²P] PPi into dNTP. If PPi binding occurs before the conformational change (FIG. 8a), then the rates of exchange and pyrophosphorolysis will be the same. The rate of the exchange reaction during catalytic cycling was identical to the rate measured by single-turnover analysis, indicating that PPi binding occurs before the conformational change that precedes chemistry (FIG. 8b).

Survey of PPi Analog-Dependent Reverse Reactions

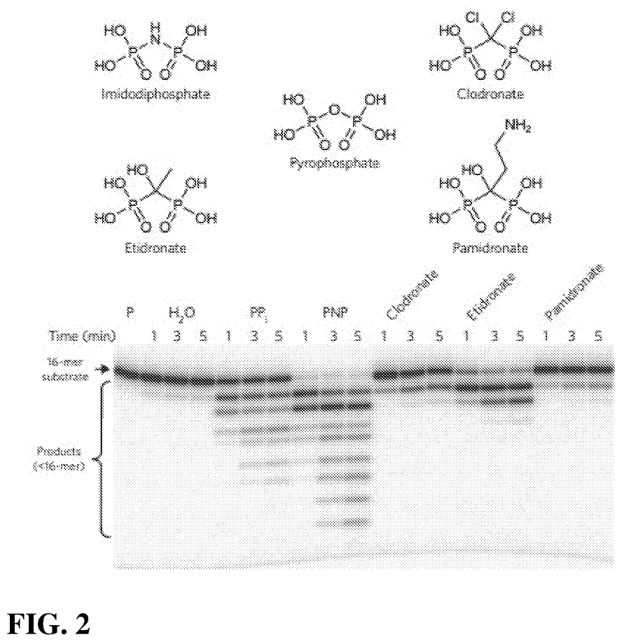

Bisphosphonates (FIG. 2) have a carbon atom in place of the bridging oxygen in PPi and are used to treat osteoporosis and bone metastasis18. We surveyed three bisphosphonates (etidronate, clodronate, and pamidronate) for their ability to serve as substrates for a reverse reaction that would generate a dNTP analog with a modified bridging atom between the β- and γ-phosphates (Pp and Py). Additionally, we examined how well imidodiphosphate, which has been used as part of ATP and GTP analogs (i.e., NMPPNP) to study adenylyl- and guanyl-using enzymes, can serve as a PPi analog for the reverse reaction.

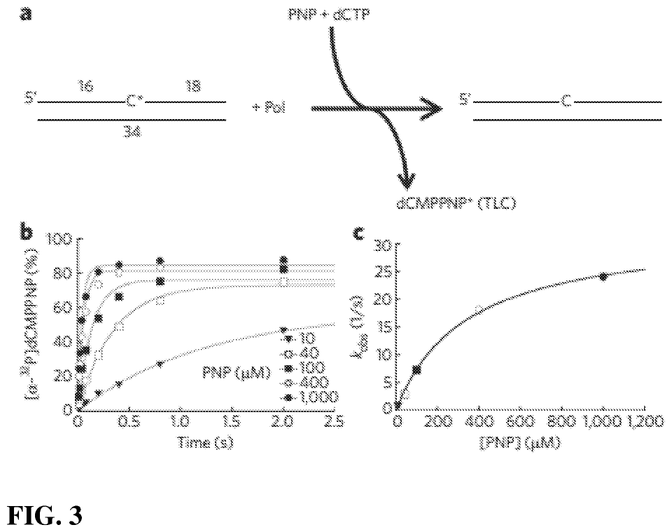

A qualitative assay to survey these analogs indicated that the bisphosphonates were moderate (for example, etidronate) to poor (clodronate and pamidronate) substrates, but PNP exhibited strong activity, continually degrading most of the DNA substrate to very short products (FIG. 2). To quantify the impact of substituting nitrogen for the bridging oxygen of PPi, single-turnover experiments were performed to determine the rate and PNP binding affinity of the enzyme (FIG. 3 and Supplementary Table 1). Although the PNP binding affinity was somewhat weaker than PPi (˜4-fold), the rate of the reverse reaction was 1,000-fold more rapid (krev ˜30 s⁻¹).

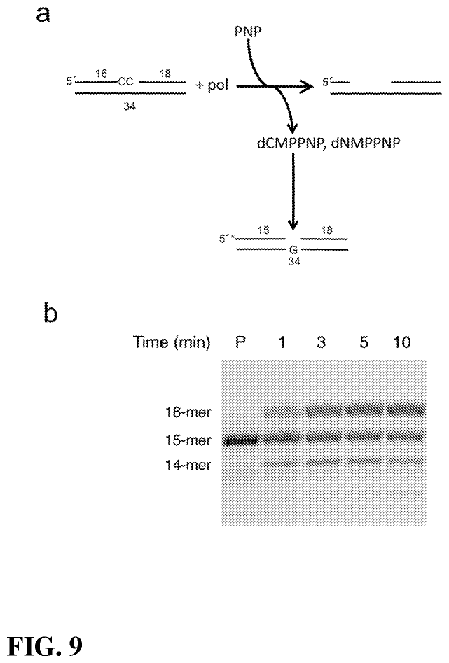

With the TLC solvent system, the dCMPPNP product migrated with a mobility similar to that expected for dCDP, as observed previously. It was also verified that the product of the PNP-initiated reverse reaction could be used in the forward reaction for a coupled DNA synthesis reaction. This reaction used two DNA substrates: unlabeled nicked DNA with a 3′-dCMP at the margin of the nick, and a single-nucleotide gapped DNA with a templating deoxyguanosine in the gap and a 5′ ³²P-labeled primer. Addition of a low concentration of PNP resulted in gap-filling DNA synthesis on the gapped DNA substrate, indicating that PNP can generate dCMPPNP, which can subsequently fill the gapped substrate (FIG. 9). Additionally, crystallographic characterization of the ternary nicked-DNA-PNP complex indicated that PNP supports a strong reverse reaction, generating a dNTP analog in which the bridging atom between Pβ and Pγ is a nitrogen atom (see below).

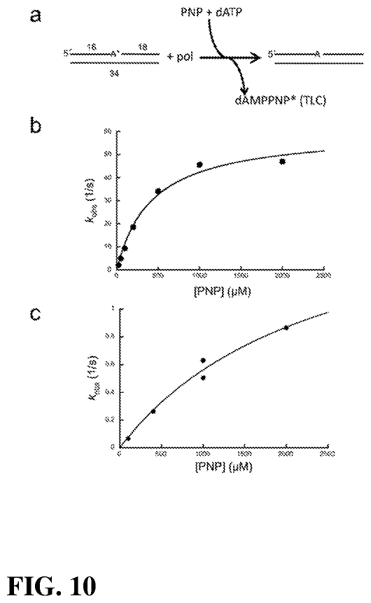

To see whether the faster rate of the observed reverse reaction initiated with PNP is limited by chemistry or a nonchemical step, the rate of removal of a 3′-terminal phosphorothioate was determined. In contrast to the lack of a phosphorothioate elemental effect on the observed reaction initiated with PPi (FIG. 7), the PNP-dependent reverse reaction was considerably slower when removing a 3′-terminal Rp-phosphorothioate nucleotide (FIG. 10). In this situation, the phosphorothioate effect was ˜30 (k_{rev(O)}/k_{rev(S)}).

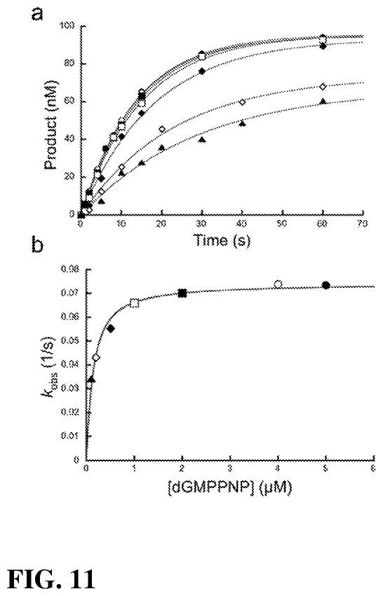

Because substitution of the bridging oxygen between Pβ and Pγ of dGTP with methylene derivatives has been shown to influence insertion efficiency the kinetics of 2′-deoxyguanosine-5′-[(β, γ)-imido]triphosphate (dGMPPNP) insertion were quantified. A single-turnover analysis indicated that the rate of insertion was strongly diminished, but binding affinity was increased (FIG. 11 and Supplementary Table 1). The decreased forward reaction coupled with the increased reverse reaction indicates a decreased chemical equilibrium relative to reaction with PPi.

Overall Equilibrium Constant

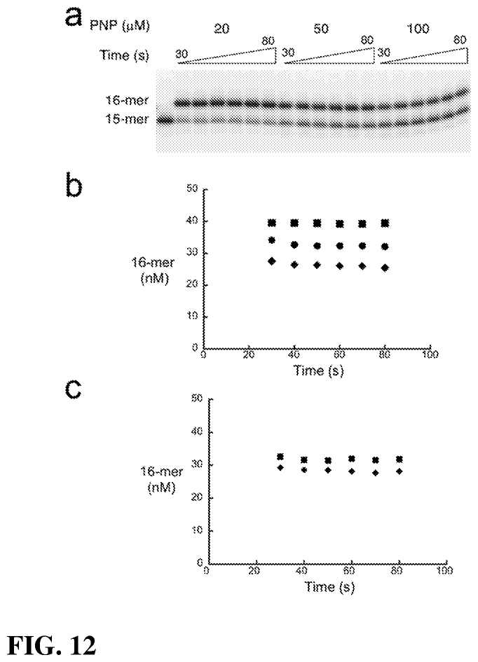

The overall equilibrium constant of enzyme-bound gapped DNA and nicked DNA was measured at several PPi concentrations. Under single-turnover conditions, 50 nM gapped DNA was incubated with Mg2+ and a low concentration of dCTP with varying concentrations of PPi. The reactions were quenched after various time intervals and reaction products separated on a sequencing gel. The substrate (gapped DNA) and product (nicked DNA) bands were quantitated, and the overall equilibrium constant calculated (FIG. 12); Keq =[nicked DNA] [PPi]/[gapped DNA]I[dCTP]. The overall equilibrium constant determined at different PPi concentrations is Keq=68,700±7,200.

In the case of the imido-analogs (dGMPPNP and PNP), the overall equilibrium was measured in a similar manner. In this case, the templating base was cytosine because the incoming nucleotide is dGMPPNP. In this situation, the concentration of dGMPPNP required to generate a substantial forward reaction, balancing the strong reverse reaction with PNP, is much higher than that needed when the bridging atom between Pβ and Pγ of the triphosphate is an oxygen atom (FIG. 12). The overall equilibrium of this reaction was considerably lower than that performed with natural substrates (Keq=1.7±0.1).

DNA Substrate Specificity for the Reverse Reaction

Insertion of a chain-terminating nucleotide or an incorrect nucleotide results in a DNA product that disrupts further DNA synthesis. This provides an opportunity to remove the 3′-terminal nucleotide by pyrophosphorolysis. The PPi-dependent removal of a 3′ mismatched nucleotide is very poor, probably due to the distorted geometry of the terminal mismatch in the polymerase active site. However, chain-terminating nucleotides are often modified in their sugar moiety, which does not perturb Watson-Crick hydrogen bonding or phosphate backbone geometry, thereby providing a good substrate for an unblocking reaction (i.e., ‘removal by reversal’).

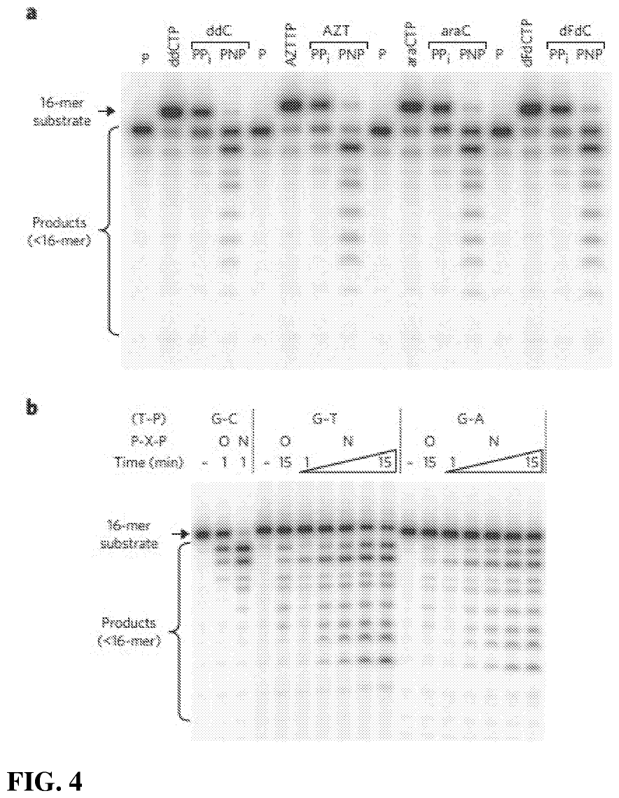

Pyrophosphate- and PNP-dependent removal of chain-terminating nucleotides is shown in FIG. 4. The DNA substrates were prepared in situ starting with single-nucleotide gapped DNA with a 5′ ³²P-labeled primer strand. An excess (relative to DNA) of chainterminating nucleoside triphosphate was added to the gapped DNA substrate and incubated with pol β to generate a nicked DNA substrate with a 3′ chain-terminating nucleotide. Pyrophosphate or PNP was added, and shortening of the labeled DNA primer strand was monitored. The chain-terminating nucleotides were removed by a reverse reaction, which occurred more rapidly when PNP was substituted for PPi.

In contrast to Watson-Crick base-paired primer termini, mismatches at the primer terminus are not good substrates for pyrophosphorolysis11. However, substituting PNP for PPi resulted in substantial removal of the mismatch (FIG. 4). Additionally, it appears that a G-T mismatch (template-primer) was removed more rapidly than a G-A terminus.

Time-Lapse Crystallography of Pyrophosphorolysis

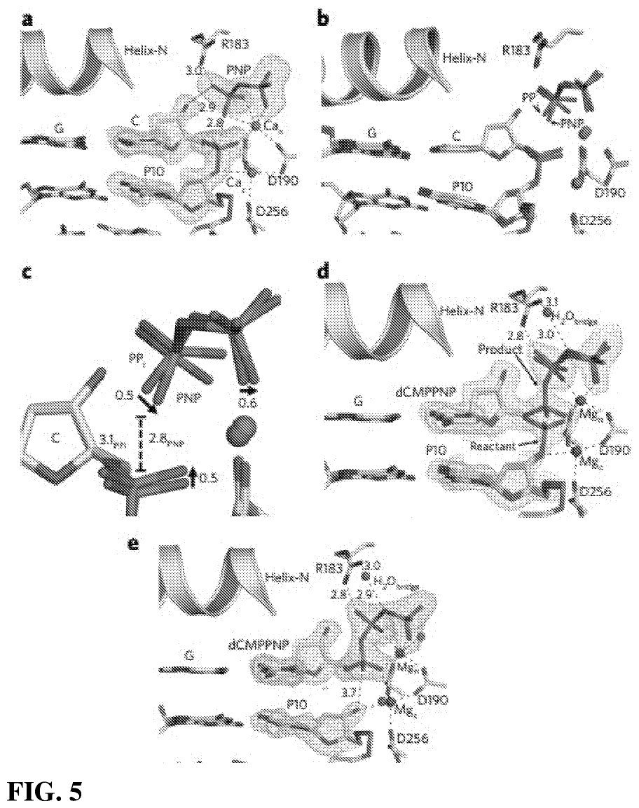

To analyze the robust nature of the reverse reaction in molecular detail, time-lapse crystallography was performed. In this approach, crystals of binary DNA complexes are soaked with substrates or metals to initiate the chemical reaction, and the reaction was stopped at time intervals by rapid freezing. The structure is then determined to identify the progress of the reaction and capture unique molecular aspects along the reaction path. Although this has been accomplished for the forward DNA synthesis reaction, it was not successful in initiating pyrophosphorolysis; i.e., PPi binding did not generate dNTP. This outcome is probably due the unfavorable chemical equilibrium. Because PNP initiated a strong reverse reaction, PNP-Ca2+ was added to crystals of binary pol β-nicked DNA complexes. Because Ca2+ is catalytically inert, PNP binding resulted in a closed precatalytic ternary complex (pol-DNAnicked-PNP), with two Ca2+ ions positioned in the metal-binding sites necessary for the forward DNA synthesis reaction (Supplementary Table 2 and FIG. 5a). The ternary PNP-Ca2+ complex was compared to the ternary PPi product complex generated with Mg2+(PDB 4KLO; FIG. 5b).

Although the structures are globally similar (r.m.s. deviation=0.29 Å over 326 Ca atoms), there were several notable, subtle differences. The PNP structure included two Ca2+ ions, in the catalytic and nucleotide metal-binding sites, whereas the PPi structure has a single Mg2+ ion bound to the nucleotide metal site and a Na+ bound in the catalytic metal site. Additionally, the precise position of the nonbridging oxygens of PNP are shifted ˜0.5 Å relative to that observed with PPi (FIG. 5c). This modest repositioning moves the attacking oxygen on PNP 0.3 Å nearer to the phosphate of the leaving group as compared to PPi (distances of 2.8 and 3.1 Å, respectively).

Soaking the crystals in a solution containing Mg2+ resulted in Mg2+ exchange for the Ca2+ ions. After a short time, the crystals were flash frozen and diffracted to 2.0 Å. Occupancy refinement indicated that approximately 40% of the complexes had undergone a reverse reaction. The catalytic and metal-binding sites contained Mg2+ ions, as deduced by coordination distances and geometry. Additionally, a unique water molecule serves as a ‘bridging’ molecule between Arg183 and the nitrogen between Pβ and Pγ of dCMPPNP (FIG. 5d). Other Mg2+ soaks resulted in complete turnover of the crystallographic complexes with product dCMPPNP bound in the closed polymerase complex. Notably, Mg2+ still occupies the catalytic metal site without apparent DNA synthesis activity. In this case, the distance between 03′ (primer terminus) and Pu (dCMPPNP) is 3.7 Å, compared to the 3.4 Å observed with deoxyuridine-5′-[(β, γ)-imido] triphosphate (PDB 2FMS; FIG. 5e).

Pyrophosphorolysis has been suggested to play a role in DNA polymerase fidelity and HIV-1 reverse transcriptase, as well as mitochondrial DNA polymerase γ sensitivity to chain-terminating nucleoside drugs. DNA polymerases that stall after insertion of a chain-terminating or aberrant nucleotide can utilize pyrophosphorolysis to remove this impediment, whereas DNA polymerases with a proofreading 3′-5′ exonuclease could employ the hydrolytic excision activity to remove the terminal nucleotide. In this latter case, a nucleoside monophosphate is produced instead of the triphosphate.

A better understanding of the reverse reaction is essential to define the overall reaction that will impact or modulate these proposed activities, and is a pre-requisite for rational drug design. In this respect, PPi analogs can inhibit the forward or reverse reaction, whereas others that enhance the reverse reaction can decrease the overall forward reaction.

The oversimplified general scheme for DNA polymerase single nucleotide insertion (FIG. 1d) serves as a useful outline for discussing and interpreting kinetic and structural observations. It does not include several key steps that can have substantial impact on activity such as catalytic metal binding and additional conformational adjustments that would impact the distribution of the enzyme-ligand complexes. The identities of the pre- and postchemistry conformational change steps are also not known. However, intensive structural characterization of a wide variety of DNA polymerases in different liganded states indicates that there are protein and substrate conformational adjustments upon ligand binding. These changes range from large enzyme subdomain motions (for example, T7 DNA polymerase) to subtle loop and side chain adjustments (for example, pol μ). Pol β-DNA binary complexes (nicked or gapped DNA) transition to closed complexes when they bind PPi or dNTP.

This modification involves repositioning of the carboxyl-terminal N-subdomain (‘fingers’ of right-handed DNA polymerases) to make intimate contacts with substrates and products. Thus, the opening and closing of the N-subdomain will be used in the context of the conformational changes (FIG. 1d).

Substrate and protein conformational adjustments play an important role in facilitating a commitment to high-fidelity DNA synthesis by sequestering the correct nucleoside triphosphate (large K3, FIG. 1d) and aligning catalytic atoms31. In addition, rapid decomposition of the ternary product complex through a two-step reaction in which a post-chemistry conformational change (large K5, FIG. 1d) facilitating rapid PPi release also commits the reaction forward. While a two-step dNTP binding mechanism is well established, the impacts of post-chemistry conformational changes and pyrophosphorolysis have received less attention. To analyze kinetic steps that occur after nucleotide insertion, the reverse reaction was characterized.

DNA polymerases have evolved to replicate DNA while deterring the reverse nucleic-acid-degrading pyrophosphorolysis reaction. This is partly due to use of a highly charged active site that ‘tunes’ natural substrates for DNA synthesis. Experimental estimates for the equilibrium constant with A- and B-family proofreading DNA polymerases (exo mutants) are ˜5,000. For pol β(X family), which lacks a proofreading activity, the equilibrium constant determined from the equilibrium concentration of enzyme-boundsubstrates and products is >10-fold higher than these reported values. This greater commitment to the forward reaction could be partly due to rapid catalytic metal dissociation after nucleotide insertion observed for pol β that would deter the reverse reaction. Quantum mechanics-molecular-mechanics calculations indicate that this metal is required for pyrophosphorolysis. Additionally, post-catalytic active site water penetration leads to the loss of nucleotide metal coordination with PPi, thereby initiating product dissociation, which would also deter pyrophosphorolysis.

DNA pol pyrophosphorolysis is slow (krev ˜0.03 s-1), as measured by single-turnover analysis (enzyme >DNA, no catalytic cycling) as well as by an exchange reaction that measures the movement of radiolabel from PPi to dNTP during alternating nucleotide insertion and removal (FIG. 8).

The lack of a thio-elemental effect for pyrophosphorolysis is consistent with a rate-limiting nonchemical conformational change preceding pyrophosphorolysis (FIG. 7a). With an unfavorable equilibrium constant after nucleotide insertion chemistry (large K₅), the observed rate for pyrophosphorolysis would underestimate the intrinsic rate (k₋₄, FIG. 1d) because the productive ternary product complex would only be a fraction of the total enzyme product (DNA+1-PPi) complexes.

By employing nucleoside triphosphates that have modified leaving groups (i.e., bridging β, γ-methylene derivatives), nucleotide insertion was shown to be strongly dependent on leaving group acidity (lower acidity resulted in decreased insertion), suggesting that bond breaking is at least partially rate limiting. The acidity of β, γ-imido-modified nucleoside triphosphates are lower than that of their natural counterparts37. In agreement with methylene substitutions, the insertion of dGMPPNP is diminished by two orders of magnitude, whereas the observed reverse reaction with PNP is increased by three orders of magnitude (Supplementary Table 1), suggesting that the overall equilibrium is altered ˜105-fold.

Substitution of sulfur with a nonbridging oxygen atom on Pu (dNTP) provides valuable mechanistic details for the polymerase catalyzed reactions. Sulfur substitution for a nonbridging oxygen on Pα should make this phosphate less susceptible to nucleophilic attack (sulfur is less electronegative than oxygen), thereby reducing the observed rate of reaction in cases where chemistry is the sole rate-limiting step. Although there was no thio-elemental effect with the PPi-initiated reverse reaction, a substantial effect (k_{rev(O)}/k_{rev(S)}˜30; FIG. 10) was observed with the PNP-initiated reaction. The more rapid rate must reflect a substantial increase in the rate of the conformational change that precedes the reverse reaction (k-5) so that it is no longer rate limiting. The water-mediated hydrogen bonding observed between the imido moiety and Arg183 may facilitate this step (FIG. 5d,e). A similar hydrogen bonding pattern has been reported in a pol β ternary complex with gapped DNA and thymidine-5′-[(α, β)-methyl:(β, γ)-imido]triphosphate (TMPCPNP).

Time-lapse crystallographic characterization of the forward reaction for pol β and pol η (ref. 40) identified an adjunct divalent metal cation coordinating reaction products (i.e., inserted dNMP and PPi). It was proposed that this metal lowers the activation barrier for the insertion reaction (i.e., increases k₄). In contrast, computational studies with pol are consistent with a role for this metal in deterring the pyrophosphorolysis reaction (i.e., decreases k₋₄). Consistent with the latter interpretation, a closed pol β ternary product complex can be formed with nicked DNA and PPi with an adjunct metal that does not undergo pyrophosphorolysis (i.e., no dNTP formation). In addition, we have been unable to solve the structure of a closed binary nicked DNA complex, consistent with rapid PPi release occurring after subdomain opening.

Notably, the ability to structurally observe the reverse reaction in the closed complex with PNP, but not PPi, indicates that the internal chemical Equilibrium is dramatically decreased when PPi is substituted with PNP. Importantly, the adjunct product metal that could interfere with the reverse reaction is not observed. The strong thio-elemental effect measured with the PNP-dependent reaction indicates that chemistry is now rate limiting. Thus, at least two steps (FIG. 1d, steps 4 and 5) have been altered to dramatically decrease the equilibrium constant. Upon binding PNP, pol β must close rapidly, forming the activated ternary complex (k₋₅>k₋₄). The ability to structurally capture the product complex (i.e., with dCMPPNP and one-nucleotide gapped DNA) of the PNP-dependent reverse reaction with magnesium in the catalytic and nucleotide binding sites suggests that the chemical equilibrium constant (K₄) is substantially less than 1. If the measured single-turnover rates are taken as the intrinsic rate constants for this step, then K₄=0.003.

Since Keq is 1,000-fold greater than this resulting K₄, surrounding equilibria must pull the DNA synthesis reaction forward. The distance between the newly formed primer terminus (O3′) and Pu of dCMPPNP (3.7 Å; FIG. 5e) is substantially greater than that observed in a precatalytic complex for the forward reaction trapped with a nonhydrolyzable nucleotide analog (3.4 Å). This increased distance may, in part, account for the diminished rate of nucleotide insertion.

Pyrophosphate-dependent primer terminus removal is considerably better with a matched than with a mismatched terminus. Although PNP improves mismatch removal, it is not as good as that seen with a matched terminus (FIG. 4b), indicating that a well-positioned primer terminus is required for optimal activity. The observation that pol λ can remove a misinserted dAMP opposite 8-oxo-deoxyguanosine (8-oxo-dG) through pyrophosphorolysis is consistent with this idea8. In this context, the mismatch mimics an A-T base pair wherein 8-oxo-dG is in a syn conformation and there is Hoogsteen base pairing with adenine. With natural substrates, resistance to chain-terminating nucleotides is minimized through a post-insertion conformational change that pulls the reaction forward (FIG. 1d, large K₅). The reversal of this conformational change (k₋₅) limits pyrophosphorolysis. In instances in which pyrophosphorolysis is elevated, drug resistance can be attributed to an altered post-nucleotide insertion nonchemical step.

The molecular identity of this step is unknown, but has often been attributed to subdomain repositioning (opening and closing) known to occur with HIV-1 reverse transcriptase, pol γ, and pol β. Imido substitution for the β, γ-bridging oxygen in the incoming nucleoside triphosphate and PPi strongly diminished the favorable equilibrium for DNA synthesis by decreasing the forward rate and hastening the reverse reaction (Supplementary Table 1). This occurs by altering conformational and chemical equilibria. Accordingly, the product of the reverse reaction (dGMPPNP) is a good inhibitor of the forward reaction (i.e., binds tightly and is inserted slowly). Although PNP-dependent removal of chain-terminating nucleotides is substantially better than that with PPi (FIG. 4a), the net result would be very low DNA polymerase activity (i.e., inhibition). Importantly, the equilibrium for the overall reaction is sensitive to the nature of the DNA synthesis leaving group, indicating that the chemistry of the terminal phosphates of an incoming nucleotide influences both chemical and conformational equilibria.

Exemplary Methods

Materials. Human pol β was expressed and purified44. Clodronate, etidronate, imidodiphosphate, pamidronate, and pyrophosphate were from Sigma-Aldrich. The β,γ-imido modified nucleoside triphosphate analog, 2′-deoxyguanosine 5′-(β, γ)-imido]triphosphate (dGMPPNP), was from Jena Bioscience. Chainterminating nucleoside triphosphates: ddCTP was from GE Healthcare; 3′-azido-2′,3′-dideoxythymidine triphosphate (AZTTP) and arabinofuranosylcytosine triphosphate (araCTP) were from TriLink BioTechnologies; and gemcitabine (dFdCTP) was obtained from Jena Bioscience. [α-³⁵S] dATP, [α-³²P] dCTP, and [³²P] PPi were from PerkinElmer. Polyethyleneimine (PEI) cellulose thin-layer chromatography (TLC) plates containing a fluorescent indicator were purchased from EMD Millipore.

Reaction buffer. All kinetic measurements were performed in a buffer containing 50 mM MES, 25 mM Tris, 25 mM ethanolamine (pH 7.5 adjusted at 37° C.), 100 mM KCl, 10 mM MgCl2 supplemented with 10% glycerol, 100 μg/ml bovine serum albumin, 1 mM DTT, and 0.1 mM EDTA.

Product separation. Changes in the length of a 5′-labeled primer strand were visualized and resolved on 16% denaturing polyacrylamide gels. The gel was scanned using a phosphorimager in fluorescence mode to visualize 6-carboxyfluorescein (6-FAM)-labeled oligonucleotides. Radiolabeled oligonucleotides were detected after exposing a dried gel to a phosphor screen.

Reverse reaction products were also separated on PEI cellulose TLC plates. Unless otherwise noted, the plates were developed in 0.2 or 0.3 M NaPi, pH 7.0. TLC of 35S-labeled reverse reaction products was performed in buffer containing 10 mM β-mercaptoethanol.

DNA preparation. Single nucleotide gapped DNA substrates containing a 5′-6-FAM label were prepared as detailed previously45. Nicked DNA substrates used to qualitatively monitor the reverse reaction were prepared as follows. Briefly, a 16-mer oligonucleotide primer was radiolabeled at the 5′-end with [γ-³²P] ATP and Optikinase. Unincorporated [γ-³²P] ATP was removed using a BioSpin 6 column. The 5′-labeled primer (1 equivalent) was mixed with 1.2 equivalents of 34-mer template and 18-mer downstream oligonucleotide containing a 5′-PO₄ group. Annealing was performed in a PCR thermocycler.

Oligonucleotides were denatured at 95° C. for 5 min followed by slow cooling (1° C./min) to 10° C. The following sequences were used to construct the nicked DNA substrates with a matched or mismatched primer terminus; primer, 5′-CTG CAG CTG ATG CGC Y-3′ (SEQ ID NO: 1), where Y denotes A, C or T; downstream oligonucleotide, 5′-GTA CGG ATC CCC CGG GTA C-3′ (SEQ ID NO: 2); template strand, 5′-GTA CCC GGG GAT CCG TAC XGC GCA TCA GCT GCA G-3′ (SEQ ID NO: 3), where X denotes G.

PNP-induced gap-filling reaction. Pol β (5 μM) was pre-incubated with 2.5 μM nicked DNA and 20 M PNP in reaction mixture without Mg2+. This was mixed (1:1, v/v) with a solution with 20 mM MgCl2 and incubated at 37° C. in reaction buffer. Following mixing, the final concentrations were 2.5 μM pol β, 1.25 μM nicked DNA, 10 μM PNP and 10 mM MgCl2. After 10 min, an aliquot was mixed (4:1, v/v) with a solution containing 2.5 μM single-nucleotide gapped DNA (G in the gap) with a 5′-6-FAM labeled 15-mer primer and 10 mM MgCl2.

Aliquots (10 μl) were removed at various times and quenched in an equal volume of 0.3 M EDTA, pH 8.0. Reaction substrates and products were separated on 16% denaturing polyacrylamide gels and visualized by phosphorimagery.

Preparation of 3′-³²P- or ³⁵S-labeled nicked DNA substrates. DNA polymerase β was used to fill a 1-nucleotide gapped DNA substrate with either [³²P] dCTP or [³⁵S] dATP to create a 3′-radiolabeled nicked DNA substrate. The reaction mixture contained 50 mM Tris-Cl, pH 7.4 (37° C.), 100 mM KCl, 10 mM MgCl2, 1 mM DTT, 2.5 μM gapped DNA, 5 M [³²P] dCTP or [³⁵S] dATP. The single-nucleotide DNA substrate was similar to the nicked substrate described above, except the primer strand was one nucleotide shorter (3′-nucleotide deleted). Gap filling was initiated by addition of pol β and incubated at 37° C. for 5-10 min. The reaction was quenched by addition of 0.5 M EDTA (0.1 vol). To remove enzyme and unincorporated nucleotides, the mixture was extracted with phenol-chloroform-isoamyl alcohol (25:24:1) followed by two passages through BioSpin 6 columns. Aliquots of the labeling reaction were removed before and following the extraction and removal steps to determine final DNA substrate concentration. Pre- and post-aliquots (1 μl) were spotted onto PEI cellulose plates and developed in 0.375 M KH₂PO₄, pH 4.0. The ratio (post/preextraction) was used to correct the initial DNA concentration for loss or dilution of substrate.

Reverse reaction assay. Pol β (1 μM) was pre-incubated with 100 nM nicked DNA containing either a matched or mismatched primer terminus for 5 min at 37° C. in reaction buffer. A solution with 20 mM MgCl2 containing 2 mM PPi or pyrophosphate analog in reaction buffer was used to initiate the reaction. Following mixing, the final concentrations were 500 nM pol β, 50 nM nicked DNA, 10 mM MgCl2, and 1 mM PPi or pyrophosphate analog. Aliquots (5 or 10 μl) were removed at various times and quenched in an equal volume of 0.3 M EDTA, pH 8.0. Reaction substrates and products were separated and visualized as described above.

The removal of a terminated primer terminus by the reverse reaction required enzymatic synthesis of the nicked DNA substrate. A pre-incubated mixture of 4 μM pol and 0.4 μM one-nucleotide gapped DNA was mixed 1:1 (v/v) with 20 mM MgCl2 and 0.2 μM various triphosphates of chain-terminating nucleotides (ddCTP, AZTTP, araCTP, or dFdCTP). The gap-filling reaction proceeded at 37° C. for 10-20 min to generate a terminated nicked DNA substrate. An aliquot was removed and quenched to verify complete gap filling (16-mer). The reverse reaction was initiated by addition of an equal volume of 10 mM MgCl2 and 250 μM PPi or PNP. An aliquot (time=3 min) was removed, quenched, and analyzed on a denaturing gel.

The removal of a terminal mismatch by a reverse reaction was followed by incubation of 1 M Pol with 100 nM 5′-³²P-labeled nicked DNA substrate with a matched (G-C) or mismatched (G-A or G-T) primer terminal base pair for 5 min at 37° C. This was mixed with a solution of 20 mM MgCl2 and 2 mM PPi or PNP (1:1, v/v) to initiate the reaction. Reactions were quenched at various time intervals with addition of an equal volume of 0.3 M EDTA and substrate and reverse reaction products separated on a denaturing gel.

Kinetic parameters for the reverse reaction were determined under single turnover conditions (E/DNA=10) at 37° C. Pol β (1 μM) was pre-incubated with 100 nM 3′-³²P-labeled primer in nicked DNA with various concentrations of PPi or PNP in reaction buffer. Time courses were initiated by mixing with an equal volume of a 20 mM MgCl2 and 50 μM dNTP trap solution in reaction buffer. The dNTP trap prevents re-insertion of radiolabeled product dNTP and corresponds to the identity of the nucleotide triphosphate produced during the reaction. Initiation of the reaction was performed by manual mixing, in the case of pyrophosphorolysis, or rapid mixing using a Kintek RQF-3 with PNP. EDTA (0.1 or 0.2 M) was used as the quenching agent. Substrates and products were resolved by TLC in either 0.2 or 0.3 M NaPi, pH 7.0 buffer.

Pyrophosphate exchange assay. Pol β (2.5 μM) was pre-incubated with 500 nM unlabeled nicked DNA substrate containing a matched primer terminal base pair (G-C, template-primer) in reaction buffer with 20 mM MgCl2 and manually mixed (1:1, v/v) with a prewarmed solution of reaction buffer, 2 mM [³²P] PPi, and 100 μM dCTP. Aliquots were withdrawn at various time points and quenched with 1 vol. of 0.3 M EDTA. Quenched reactions mixtures were applied to PEI cellulose plates and developed in 0.3 M potassium phosphate buffer, pH 8.0. Plates were scanned followed by quantitation using a phosphorimager and ImageQuant software.

Gap filling DNA synthesis kinetic assay. To measure the rate of the first insertion (kpol) and apparent equilibrium nucleotide dissociation constant (K_{d}), single-turnover kinetic assays (enzyme/DNA=10) were performed as described previously. Briefly, a pre-incubated solution of enzyme and DNA was rapidly mixed with various concentrations of MgCl₂ and dGMPPNP using a Kintek RQF-3 rapid quench-flow. Reactions were quenched with 0.25 M EDTA.

Kinetic analysis. Single-turnover time courses were fit to a single exponential equation to yield the first-order rate constants (k_{obs}) at a given concentration of GMPPNP, PPi or PNP. Under these conditions, k_{obs} was dependent on the concentration of substrate. A secondary plot of the concentration dependence of kobs was hyperbolic and fitted by nonlinear least-squares method to equation (1) where kmax is the intrinsic rate constant for the step limiting the first nucleotide insertion (forward reaction) or removal (reverse reaction).

k_{obs}=kₘₐₓ[S]/(Kd[S]) (1)

where S=dGMPPNP, PPi, or PNP. For the insertion of dGMPPNP, the secondary plot was fit to a quadratic equation (equation (2)) due to its high affinity relative to the enzyme concentration.

k_{obs}=(kₚₒₗ)(((K_{d}+[dGMPPNP]+[E_{DNA}])((K_{d}+[dGMPPNP]+[E_{DNA}])²)−(4[dGMPPNP][E_{DNA}]))^{0.5})/2[E_{DNA}] (2)

Data points, time and ligand concentrations, were selected to provide full coverage; i.e., multiple points were collected below and above reaction half-times (≥6 time points) and ligand binding affinities (≥5 concentrations), respectively. Unless noted, kinetic constants represent best-fit parameters and their standard error.

Overall Equilibrium Constant Determination.

A mixture of 500 nM pol β with single-nucleotide gapped DNA (pol/DNA=10; templating G or C) containing various concentrations of PPi (500-2,000 μM) or PNP (20, 50, 100 μM) was mixed with an equal volume of 20 mM MgCl2 containing 60-100 nM dCTP or 50 μM dGMPPNP and incubated at 37° C. for various time intervals. Aliquots (10 μl) were withdrawn at various times and quenched with an equal volume of 0.3 M EDTA. The reactions were quenched after 10-80 s and reaction products separated on a sequencing gel. The substrate (gapped DNA) and product (nicked DNA) bands were quantitated, and the overall equilibrium constant calculated; Keq=[nicked DNA][PPi]/[gapped DNA][dCTP] or [nicked DNA] [PNP]/[gapped DNA][dGMPPNP]. The mean and standard error for 6 independent determinations are reported in the text.

Structure Determination.

Binary complex crystals with nicked DNA were grown as previously described43. The time-lapse crystallography was performed as before11 and is briefly summarized here. Binary pol β/DNA complex crystals were first transferred to a cryosolution containing 15% ethylene glycol, 50 mM imidazole, pH 7.5, 20% PEG3350, 90 mM sodium acetate, 2 mM PNP and 50 mM CaCl₂) for 1 h. These ground state (GS) ternary complex crystals were then transferred to a cryosolution containing 200 mM MgCl2 for varying times. All reactions were stopped by freezing the crystals at 100K before data collection at the home source, 1.54 Å, or the Advanced Photon Source, 1.0 Å (Argonne National Laboratory). In house data collection was done on a SATURN92 CCD detector system mounted on a MiraMax-007HF rotating anode generator. This allows for anomalous data detection after phasing by molecular replacement. Remote data collection was done at Southeast Regional Collaborative Access Team (SER-CAT) BM-22 beamline at the Advanced Photon Source (Argonne National Laboratory) with the MAR225 area detector. Data were processed and scaled using the HKL2000 software package47. Initial models were determined using molecular replacement with the open binary (PDB ID 3ISB) or closed ternary (PDB ID 2FMS) structures of pol β and all Rfree flags were taken from the starting model. Refinement was carried out using PHENIX and model building using Coot. The metal-ligand coordination restraints were generated by ReadySet (PHENIX) and not used until the final rounds of refinement. Partial catalysis models were generated with both the reactant and product species and occupancy refinement was performed. The structural figures were prepared in Pymol (Schrödinger, LLC) and all density maps were generated after performing simulated annealing. Ramachandran analysis determined 100% of nonglycine residues lie in allowed regions and at least 97% in favored regions.

In any of the embodiments described herein, the method comprises dividing (b) into at least one additional secondary reaction including a second site-specific secondary primer complementary to a second site-of interest that may be present within the primary amplicon and defines a second site of interest within the region of interest.

Those skilled in the art will recognize, or be able to ascertain using no more than routine experimentation, many equivalents to the specific embodiments of the invention described herein. Such equivalents are intended to be encompassed by the following claims. It is understood that the detailed examples and embodiments described herein are given by way of example for illustrative purposes only, and are in no way considered to be limiting to the invention. Various modifications or changes in light thereof will be suggested to persons skilled in the art and are included within the spirit and purview of this application and are considered within the scope of the appended claims. For example, the relative quantities of the ingredients may be varied to optimize the desired effects, additional ingredients may be added, and/or similar ingredients may be substituted for one or more of the ingredients described. Additional advantageous features and functionalities associated with the systems, methods, and processes of the present invention will be apparent from the appended claims. Moreover, those skilled in the art will recognize, or be able to ascertain using no more than routine experimentation, many equivalents to the specific embodiments of the invention described herein. Such equivalents are intended to be encompassed by the following claims.

### REFERENCES

The following references are incorporated herein by reference in their entirety for all purposes.
1. Bebenek, K. & Kunkel, T. A. Functions of DNA polymerases. Adv. Protein Chem. 69, 137-165 (2004).
2. Deutscher, M. P. & Kornberg, A. Enzymatic synthesis of deoxyribonucleic acid. 28. The pyrophosphate exchange and pyrophosphorolysis reactions of deoxyribonucleic acid polymerase. J. Biol. Chem. 244, 3019-3028 (1969).
3. Parsons, J. L., Nicolay, N. H. & Sharma, R. A. Biological and therapeutic relevance of nonreplicative DNA polymerases to cancer. Antioxid. Redox Signal. 18, 851-873 (2013).
4. McKenna, C. E., Kashemirov, B. A., Peterson, L. W. & Goodman, M. F. Modifications to the dNTP triphosphate moiety: from mechanistic probes for DNA polymerases to antiviral and anti-cancer drug design. Biochim. Biophys. Acta. 1804, 1223-1230 (2010).
5. Smith, A. J., Meyer, P. R., Asthana, D., Ashman, M. R. & Scott, W. A. Intracellular substrates for the primer-unblocking reaction by human immunodeficiency virus type 1 reverse transcriptase: detection and quantitation in extracts from quiescent- and activated-lymphocyte subpopulations. Antimicrob. Agents Chemother. 49, 1761-1769 (2005).
6. Urban, S., Urban, S., Fischer, K. P. & Tyrrell, D. L. Effcient pyrophosphorolysis by a hepatitis B virus polymerase may be a primer-unblocking mechanism. Proc. Natl. Acad. Sci. USA 98, 4984-4989 (2001).
7. Hanes, J. W. & Johnson, K. A. A novel mechanism of selectivity against AZT by the human mitochondrial DNA polymerase. Nucleic Acids Res. 35, 6973-6983 (2007).
8. Crespan, E., Maga, G. & Hubscher, U. A new proofreading mechanism for lesion bypass by DNA polymerase-D. EMBO Rep. 13, 68-74 (2011).
9. Beard, W. A. & Wilson, S. H. Structure and mechanism of DNA polymerase 3. Biochemistry 53, 2768-2780 (2014).
10. Perera, L., Beard, W. A., Pedersen, L. G. & Wilson, S. H. Chapter Four—Applications of quantum mechanical/molecular mechanical methods to the chemical insertion step of DNA and RNA polymerization. Adv. Protein Chem. Struct. Biol. 97, 83-113 (2014). Freudenthal, B. D., Beard, W. A., Shock, D. D. & Wilson, S. H. Observing a DNA polymerase choose right from wrong. Cell 154, 157-168 (2013).
12. Kirby, T. W. et al. Metal-induced DNA translocation leads to DNA polymerase conformational activation. Nucleic Acids Res. 40, 2974-2983 (2012).
13. Das, K. et al. Conformational states of HIV-1 reverse transcriptase for nucleotide incorporation vs pyrophosphorolysis-binding of foscarnet. ACS Chem. Biol. 11, 2158-2164 (2016).
14. Sawaya, M. R., Prasad, R., Wilson, S. H., Kraut, J. & Pelletier, H. Crystal structures of human DNA polymerase β complexed with gapped and nicked DNA: evidence for an induced fit mechanism. Biochemistry 36, 11205-11215 (1997).
15. Eckstein, F. Nucleoside phosphorothioates. Annu. Rev. Biochem. 54, 367-402 (1985).
16. Vande Berg, B. J., Beard, W. A. & Wilson, S. H. DNA structure and aspartate 276 influence nucleotide binding to human DNA polymerase β. Implication for the identity of the rate-limiting conformational change. J. Biol. Chem. 276, 3408-3416 (2001).
17. Liu, J. & Tsai, M. D. DNA polymerase β: pre-steady-state kinetic analyses of dATP α S stereoselectivity and alteration of the stereoselectivity by various metal ions and by site-directed mutagenesis. Biochemistry 40, 9014-9022 (2001).
18. Lipton, A. Emerging role of bisphosphonates in the clinic-antitumor activity and prevention of metastasis to bone. Cancer Treat. Rev. 34 (Suppl. 1), S25-S30 (2008).
19. Rozovskaya, T. et al. Pyrophosphate analogues in pyrophosphorolysis reaction catalyzed by DNA polymerases. FEBS Lett. 247, 289-292 (1989).
20. Penningroth, S. M., Olehnik, K. & Cheung, A. ATP formation from adenyl-5□-yl imidodiphosphate, a nonhydrolyzable ATP analog. J. Biol. Chem. 255, 9545-9548 (1980).
21. Oertell, K. et al. Transition state in DNA polymerase α catalysis: rate-limiting chemistry altered by base-pair configuration. Biochemistry 53, 1842-1848 (2014).
22. Oertell, K. et al. Effect of β,-CHF- and β,γ-CHCl-dGTP halogen atom stereochemistry on the transition state of DNA polymerase β. Biochemistry 51, 8491-8501 (2012).
23. Sucato, C. A. et al. Modifying the β, leaving-group bridging oxygen alters nucleotide incorporation effciency, fidelity, and the catalytic mechanism of DNA polymerase β. Biochemistry 46, 461-471 (2007).
24. Sucato, C. A. et al. DNA polymerase β fidelity: halomethylene-modified leaving groups in pre-steady-state kinetic analysis reveal differences at the chemical transition state. Biochemistry 47, 870-879 (2008).
25. Batra, V. K., Beard, W. A., Pedersen, L. C. & Wilson, S. H. Structures of DNA polymerase mispaired DNA termini transitioning to pre-catalytic complexes support an induced-fit fidelity mechanism. Structure 24, 1863-1875 (2016).
26. Vaisman, A., Ling, H., Woodgate, R. & Yang, W. Fidelity of Dpo4: effect of metal ions, nucleotide selection and pyrophosphorolysis. EMBO J. 24, 2957-2967 (2005).
27. Li, A., Gong, S. & Johnson, K. A. Rate-limiting pyrophosphate release by HIV reverse transcriptase improves fidelity. J. Biol. Chem. 291, 26554-26565 (2016).
28. Cruchaga, C., Ans6, E., Rouzaut, A. & Martfnez-Irujo, J. J. Selective excision of chain-terminating nucleotides by HIV-1 reverse transcriptase with phosphonoformate as substrate. J. Biol. Chem. 281, 27744-27752 (2006).
29. Yanvarev, D. V. et al. Methylene bisphosphonates as the inhibitors of HIV RT phosphorolytic activity. Biochimie 127, 153-162 (2016).
30. Balbo, P. B., Wang, E. C.-W. & Tsai, M.-D. Kinetic mechanism of active site assembly and chemical catalysis of DNA polymerase. Biochemistry 50, 9865-9875 (2011).
31. Tsai, Y.-C. & Johnson, K. A. A new paradigm for DNA polymerase specificity. Biochemistry 45, 9675-9687 (2006).
32. Dahlberg, M. E. & Benkovic, S. J. Kinetic mechanism of DNA polymerase I (Klenow fragment): identification of a second conformational change and evaluation of the internal equilibrium constant. Biochemistry 30, 4835-4843 (1991).
33. Oertell, K. et al. Kinetic selection vs. free energy of DNA base pairing in control of polymerase fidelity. Proc. Natl. Acad. Sci. USA 113, E2277-E2285 (2016).
34. Patel, S. S., Wong, I. & Johnson, K. A. Pre-steady-state kinetic analysis of processive DNA replication including complete characterization of an exonuclease-deficient mutant. Biochemistry 30, 511-525 (1991).
35. Olson, A. C., Patro, J. N., Urban, M. & Kuchta, R. D. The energetic difference between synthesis of correct and incorrect base pairs accounts for highly accurate DNA replication. J. Am. Chem. Soc. 135, 1205-1208 (2013).
36. Perera, L. et al. Requirement for transient metal ions revealed through computational analysis for DNA polymerase going in reverse. Proc. Natl. Acad. Sci. USA 112, E5228-E5236 (2015).
37. Yount, R. G. Adenylylimidodiphosphate and guanylylimidodiphosphate. Methods Enzymol. 38, 420-427 (1974).
38. Johnson, K. A. Conformational coupling in DNA polymerase fidelity. Annu. Rev. Biochem. 62, 685-713 (1993).
39. Kadina, A. P. et al. Two scaffolds from two flips: (α, β)/(β,γ) CH2/NH “Met-Im” analogues of dTTP. Org. Lett. 17, 2586-2589 (2015).
40. Nakamura, T., Zhao, Y., Yamagata, Y., Hua, Y. J. & Yang, W. Watching DNA polymerase □ make a phosphodiester bond. Nature 487, 196-201 (2012).
41. Gao, Y. & Yang, W. Capture of a third Mg2+ is essential for catalyzing DNA synthesis. Science 352, 1334-1337 (2016).
42. Vyas, R., Reed, A. J., Tokarsky, E. J. & Suo, Z. Viewing human DNA polymerase β faithfully and unfaithfully bypass an oxidative lesion by time-dependent crystallography. J. Am. Chem. Soc. 137, 5225-5230 (2015).
43. Batra, V. K. et al. Magnesium-induced assembly of a complete DNA polymerase catalytic complex. Structure 14, 757-766 (2006).
44. Beard, W. A. & Wilson, S. H. Purification and domain-mapping of mammalian DNA polymerase β. Methods Enzymol. 262, 98-107 (1995).
45. Freudenthal, B. D. et al. Uncovering the polymerase-induced cytotoxicity of an oxidized nucleotide. Nature 517, 635-639 (2015).
46. Beard, W. A., Shock, D. D., Batra, V. K., Prasad, R. & Wilson, S. H. Substrateinduced DNA polymerase β activation. J. Biol. Chem. 289, 31411-31422 (2014).
47. Otwinowski, Z. & Minor, W. Processsing of X-ray diffraction data collected in oscillation mode. Methods Enzymol. 276, 307-326 (1997).
48. Adams, P. D. et al. PHENIX: a comprehensive Python-based system for macromolecular structure solution. Acta Crystallogr. D Biol. Crystallogr. 66, 213-221 (2010).
49. Emsley, P. & Cowtan, K. Coot: model-building tools for molecular graphics. Acta Crystallogr. D Biol. Crystallogr. 60, 2126-2132 (2004).
50. Gabbara, S., and Peliska, J. A. (1996) Catalaytic activities associated with retroviral and viral polymerases. Methods Enzymol. 275, 276-310.

1. A composition comprising a pyrophosphate (PPi) analog and a pharmaceutically acceptable carrier.

2. The composition of claim 1, wherein the pyrophosphate analog is imidodiphosphate (PNP).

3. A method of treating or ameliorating the symptoms of a disease or disorder comprising administering to a patient in need thereof, an effective amount of a composition comprising a pyrophosphate (PPi) analog, wherein the composition is effective in treating or ameliorating at least one symptom of the disease or disorder.

4. The method of claim 3, wherein the pyrophosphate analog is imidodiphosphate (PNP).

5. The method of claim 4, wherein the disease or disorder is at least one of a hyperproliferative disorder or a microbial-related disease or disorder.

6. The method of claim 5, wherein the microbial-related disease or disorder is selected from the group consisting of a bacterial or viral infection.

7. The method of claim 6, wherein the viral infection is an Adenovirus infection, a Herpes simplex type 1 infection, a Herpes simplex type 2 infection, a Varicella-zoster virus infection, a Epstein-Barr virus infection, a Human cytomegalovirus infection, a Human herpesvirus type 8 infection, a Human papillomavirus infection, a BK virus infection, a JC virus infection, a Smallpox infection, a Hepatitis B infection, a Human bocavirus infection, a Parvovirus B19 infection, aHuman astrovirus infection, a Norwalk virus infection, a coxsackievirus infection, a hepatitis A virus infection, a poliovirus infection, a rhinovirus infection, a Severe acute respiratory syndrome virus infection, a Hepatitis C virus infection, a yellow fever virus infection, a dengue virus infection, a West Nile virus infection, a Rubella virus infection, a Hepatitis E virus infection, a Human immunodeficiency virus (HIV) infection, an Influenza virus infection, a Guanarito virus infection, a Junin virus infection, a Lassa virus infection, a Machupo virus infection, a Sabiá virus infection, a Crimean-Congo hemorrhagic fever virus infection, an Ebola virus infection, a Marburg virus infection, a Measles virus infection, a Mumps virus infection, a Parainfluenza virus infection, a Respiratory syncytial virus infection, a Human metapneumovirus infection, a Hendra virus infection, a Nipah virus infection, a Rabies virus infection, a Hepatitis D infection, a Rotavirus infection, an Orbivirus infection, a Coltivirus infection, or a Banna virus infection.

8. The method of claim 6, wherein the viral infection is HIV-1 infection.

9. The method of claim 3 further comprising administering an effective amount of an additional therapeutic or bioactive agent to the patient in need thereof.

10. The method of claim 9, wherein the additional therapeutic or bioactive agent is administered concurrently with the composition comprising a pyrophosphate (PPi) analog.

11. The method of claim 9, wherein the additional therapeutic or bioactive agent is administered sequentially with the composition comprising a pyrophosphate (PPi) analog.

12. The method of claim 9, wherein the additional therapeutic or bioactive agent is an antibiotic, an anti-cancer agent, an anti-inflammatory, an antimicrobial, an antiviral, an antifungal, an antipsychotic, or an anti-HIV agent.

13. The method of claim 12, wherein the additional therapeutic or bioactive agent is an anti-HIV agent.

14. The method of claim 13, wherein the anti-HIV agent is evirapine (BI-R6-587), delavirdine (U-90152S/T), efavirenz (DMP-266), UC-781 (N-[4-chloro-3-(3-methyl-2-butenyloxy)phenyl]-2methyl3-furancarbothiamide), etravirine (TMC125), Trovirdine (Ly300046.HCl), MKC-442 (emivirine, coactinon), HI-236, HI-240, HI-280, HI-281, rilpivirine (TMC-278), MSC-127, HBY 097, DMP266, Baicalin (TJN-151) ADAM-II (Methyl3′,3′-dichloro-4′,4″-dimethoxy-5′,5″-bis(methoxycarbonyl)-6,6-diphenylhexenoate), Methyl 3-Bromo-5-(1-5-bromo-4-methoxy-3-(methoxycarbonyl)phenyl)hept-1-enyl)-2-methoxybenzoate (Alkenyldiarylmethane analog, Adam analog), (5-chloro-3-(phenylsulfinyl)-2′-indolecarboxamide), AAP-BHAP (U-104489 or PNU-104489), Capravirine (AG-1549, s-1153), atevirdine (U-87201E), aurin tricarboxylic acid (SD-095345), 1-[(6-cyano-2-indolyl)carbonyl]-4-[3-(isopropylamino)-2-pyridinyl]piperazine, 1-[5-[[N-(methyl)methylsulfonylamino]-2-indolylcarbonyl-4-[3-(isopropylamino)-2-pyridinyl]piperazine, 1-[3-(Ethylamino)-2-[pyridinyl]-4-[(5-hydroxy-2-indolyl)carbonyl]piperazine, 1-[(6-Formyl-2-indolyl)carbonyl]-4-[3-(isopropylamino)-2-pyridinyl]piperazine, 1-[[5-(Methylsulfonyloxy)-2-indoyly)carbonyl]-4-[3-(isopropylamino)-2-pyridinyl]piperazine, U88204E, Bis(2-nitrophenyl)sulfone (NSC 633001), Calanolide A (NSC675451), Calanolide B, 6-Benzyl-5-methyl-2-(cyclohexyloxy)pyrimidin-4-one (DABO-546), DPC 961, E-EBU, E-EBU-dm, E-EPSeU, E-EPU, Foscarnet (Foscavir), HEPT (1-[(2-Hydroxyethoxy)methyl]-6-(phenylthio)thymine), HEPT-M (1-[(2-Hydroxyethoxy)methyl]-6-(3-methylphenyl)thio)thymine), HEPT-S(1-[(2-Hydroxyethoxy)methyl]-6-(phenylthio)-2-thiothymine), Inophyllum P, L-737,126, Michellamine A (NSC650898), Michellamine B (NSC649324), Michellamine F, 6-(3,5-Dimethylbenzyl)-1-[(2-hydroxyethoxy)methyl]-5-isopropyluracil, 6-(3,5-Dimethylbenzyl)-1-(ethyoxymethyl)-5-isopropyluracil, NPPS, E-BPTU (NSC 648400), Oltipraz (4-Methyl-5-(pyrazinyl)-3H-1,2-dithiole-3-thione), N-{2-(2-Chloro-6-fluorophenethyl]-N′-(2-thiazolyl)thiourea (PETT Cl, F derivative), N-{2-(2,6-Difluorophenethyl]-N′-[2-(5-bromopyridyl)]thiourea {PETT derivative), N-{2-(2,6-Difluorophenethyl]-N′-[2-(5-methylpyridyl)]thiourea {PETT Pyridyl derivative), N-[2-(3-Fluorofuranyl)ethyl]-N′-[2-(5-chloropyridyl)]thiourea, N-[2-(2-Fluoro-6-ethoxyphenethyl)]-N′-[2-(5-bromopyridyl)]thiourea, N-(2-Phenethyl)-N′-(2-thiazolyl)thiourea (LY-73497), L-697,639, L-697,593, L-697,661, 3-[2-(4,7-Difluorobenzoxazol-2-yl)ethyl}-5-ethyl-6-methyl(pypridin-2(1H)-thione (2-Pyridinone Derivative), 3-[[(2-Methoxy-5,6-dimethyl-3-pyridyl)methyl]amine]-5-ethyl-6-methyl(pypridin-2(1H)-thione, R82150, R82913, R87232, R88703, R89439 (Loviride), R90385, 5-2720, Suramin Sodium, TBZ (Thiazolobenzimidazole, NSC 625487), Thiazoloisoindol-5-one, (+)(R)-9b-(3,5-Dimethylphenyl-2,3-dihydrothiazolo[2,3-a]isoindol-5(9bH)-one, Tivirapine (R86183), UC-38 or UC-84.

15. A method of inhibiting the DNA synthesis reaction of HIV-1 Reverse Transcriptase in a patient comprising administering to a patient in need thereof, an effective amount of a composition comprising a pyrophosphate (PPi) analog, wherein the composition is effective in inhibiting the DNA synthesis reaction of HIV-1 Reverse Transcriptase (RT).

16. The method of claim 8, wherein the pyrophosphate analog is imidodiphosphate (PNP).


SEQUENCE LISTING (3 sequences)

SEQ ID NO: 1 - DNA, 16 bp, Artificial Sequence
  Description of Artificial Sequence Synthetic primer
ctgcagctgatgcgch

SEQ ID NO: 2 - DNA, 19 bp, Artificial Sequence
  Description of Artificial Sequence Synthetic oligonucleotide
gtacggatcccccgggtac

SEQ ID NO: 3 - DNA, 34 bp, Artificial Sequence
  Description of Artificial Sequence Synthetic oligonucleotide
gtacccggggatccgtacggcgcatcagctgcag